(19) United States
(12) Patent Application Publication
(10) Pub. No.: US 2003/0049687 A1
(43) Pub. Date: Mar. 13, 2003

(54) Novel methods for generalized comparative modeling

(76) Inventor: Jeffrey Skolnick, Creve Coeur, MO (US)

Correspondence Address: GENEFORMATICS, INC., SUITE 200, 5830 OBERLIN DRIVE, SAN DIEGO, CA, 92121, US

(21) Appl. No.: 10/113,721

(22) Filed: Mar. 30, 2002

Related U.S. Application Data
(60) Provisional application No. 60/280,592, filed on Mar. 30, 2001.

(51) Int. Cl. G01N 33/53; G06G 7/48; G06G 7/58

(52) U.S. Cl. 435/7.1; 703/11; 702/19

(57) ABSTRACT

Improved methods for generalized comparative modeling are described, as is the application of a preferred embodiment of such methods on the Fischer database of 68 probe-template pairs, a standard benchmark to evaluate threading approaches. Briefly, the invention utilizes ab initio folding (for example, a lattice protein model, SICHO (for “Side Chain Only”) near a template provided by an alignment method, for example, a threading algorithm (e.g., PROSPECTOR). These methods can be readily automated and implemented on whole genome (or proteome) scales.

### RELATED APPLICATION

This application claims the benefit of and priority to U.S. provisional patent application serial No. 60/280,592, of the same title, filed Mar. 30, 2001.

### GOVERNMENT FUNDING

The subject matter described herein was supported in part by NIH grant GM-48835 of the Division of General Medical Sciences of the National Institutes of Health. As such, the government may have certain rights in the claimed invention.

### FIELD OF THE INVENTION

The present invention relates to computational methods for determining the three-dimensional structure of one or more proteins. More specifically, this invention relates to computer-implemented methods for performing comparative model building of protein structures.

### BACKGROUND OF THE INVENTION

The following description includes information that may be useful in understanding the present invention. It is not an admission that any of the information provided herein is prior art, or relevant, to the presently claimed inventions, or that any publication specifically or implicitly referenced is prior art.

The reported completion of the sequencing of the human genome, as well as the genomes of scores of other organisms, has ushered in what has been referred to as the “post-genomic” era, where knowledge of organisms' full proteomes (i.e., the complete set of proteins, known at the level of amino acid sequence, encoded by the corresponding genome) is now possible. Despite the vast amount of nucleic acid and protein sequence information that has been generated, and continues to be generated at a torrid pace, additional challenges must be surmounted in order to maximize the value and utility of this rapidly expanding body of genomic information.

Among the primary challenges being faced is determining the three-dimensional structures of the proteins for which at least the amino acid sequences are known. Understanding protein three-dimensional structure is critical for many reasons, including for understanding protein function on the biochemical level, as structure confers function.

Understanding protein function is important not just for biochemical reasons, but also for purposes of drug discovery, as many drug discovery efforts now focus on designing compounds to interact with specific structures in proteins. See, e.g., U.S. Pat. Nos. 6,183,121, 6,303,287, 6,162,613, 6,153,579, and 6,057,119.

Traditionally, the highest quality structure determination methods have been experimental structure prediction methods based on x-ray crystallography and NMR spectroscopy. However, these and other experimental methods for determining protein structure are extremely labor, capital, and time intensive. For example, the various publicly funded structural genomics projects (Berman, et al. (2000), Nature Structural Biology, vol. 7 (11):957-959) stated initial goal is to produce 10,000 non-redundant protein structures in ten years at a cost in the tens of millions of dollars. In contrast, it is estimated that the human proteome alone comprises well in excess of 100,000 distinct proteins. Furthermore, in the 40 years that scientists have been using experimental methods to determine protein structure, to date only 10,000 or so non-redundant protein structures have been deposited in the Protein Data Bank (Berman, et al. (2000), The Protein Data Bank, Nucleic Acids Res., vol. 28:235-242).

Given the inherent limitations associated with experimental methods of protein structure determination, the computational determination of protein structure from a protein's amino acid sequence clearly has become a very urgent task for biologists (Skolnick, et al., Nat. Biotechnol., vol. 2000, no. 18:283-7). For a given protein sequence of unknown structure, currently there are three different computational approaches to structure prediction. The most welcomed situation occurs when for a query protein it is possible to find (using standard sequence comparison tools) another protein that is highly homologous (30% or higher sequence similarity) and for which the structure has been already solved experimentally (Sanchez, et al., Proteins 1997; suppl:50-8; Sanchez, et al. (2000), Nucleic Acids Res., vol. 28:250-253; Sternberg, et al. (1999), Curr. Opin. Struct. Biol., vol. 9:368-373; Alwyn, et al., Proteins 1999; suppl:30-46). In such cases, classical comparative modeling methods allow for the construction of molecular models, the accuracy of which is sometimes (depending on the sequence similarity and “completeness” of the sequence alignment) close to that of experimental methods.

Another quite typical situation is when sequence alignment methods, or threading procedures, can detect only weakly similar sequences of protein(s) of known structure (Bryant, S. H., Proteins 1996; vol. 26:172-85; Jones, D. T. (1999), J. Mol. Biol., vol. 287:797-815; Wilmanns, et al. (1995), Protein Eng., vol. 8:626-639; Skolnick, et al., Defrosting the frozen approximation: PROSPECTOR: A new approach to threading, Proteins 2001; Panchenko, et al. (2000), J. Mol. Biol., vol. 296:1319-31). Consequently, the similarity of the (unknown) three-dimensional structure of the query sequence to the template structure cannot be quantified a priori. The two structures may have identical topology; however, they may differ in the details of their loop conformations. Also, particular secondary structure elements may be of different size, and there may be different packing angles between secondary structural elements. Further, the actual structural similarity may be limited to only that part of the structure having a common structural motif (or motifs), while the remainder of the structure is completely different (Panchenko, et al. (2000), J. Mol. Biol., supra; Panchenko, et al., Proteins 1999; suppl:133-40). In the past, application of comparative modeling tools to such cases led to molecular models that differed substantially from the structure of the query protein as determined by a different method (Alwyn, et al., Proteins 1999, supra).

A third computational approach to protein structure determination occurs when the (unknown) fold of the query protein is significantly different from any known protein fold or when the existing sequence comparison and threading tools are unable to detect an appropriate structural template. In such situations, ab initio methods to protein structure prediction may be employed required (Lee, et al., Proteins 1999, suppl 3:204-208; Ortiz, et al., Proteins 1999, suppl 3:177-185; Simons, et al., Proteins 1999, suppl:171-176; Orengo, et al., Proteins 1999, 37:149-170). While progress in the field of ab initio structure prediction has been rapidly made, (as demonstrated by a number of groups during the CASP3 exercise (Third Community Wide Experiment on the Critical Assessment of Techniques for Protein Structure Prediction, Asilomar Conference Center, Dec. 13-17, 1998), successful applications are still limited to relatively small proteins with simple fold topologies (Orengo, et al., Proteins 1999, supra). Moreover, ab initio methods, even when successful, usually provide structures of low resolution. As shown elsewhere, quite often such predicted structures can be helpful in predicting the biological functions, in particular the biochemical functions, of proteins (U.S. Ser. No. 09/493,022, filed May 27, 1999; Skolnick, et al. (2000), Nat. Biotechnol., supra; Fetrow, et al. (1998), J. Mol. Biol., vol. 281:949-968; Fetrow, et al. (1998), J. Mol. Biol., vol. 282:703-711; Skolnick, et al., TIBTECH 2000, vol. 18:34-39; Zhang, et al. (1998), Fold Des., vol. 3:535-48).

As those in the art will appreciate, it would be preferred to distinguish between protein templates of differing qualities for use in calculating three-dimensional structures for amino acid sequences. For a template of high quality, it would be preferred if a probe sequence was tightly associated with it, whereas if the template is of poor quality, then the probe sequence should be loosely bound.

The instant invention represents a significant advance in overcoming limitations inherent in conventional protein modeling techniques so that useful, computationally derived models of protein structure can be calculated for proteins for which only models of moderate to low quality could be generated, if at all, as well as to further refine computational models generated by conventional modeling techniques.

### SUMMARY OF THE INVENTION

The present invention concerns new comparative modeling methods useful in generating representations of protein structures. Thus, a first aspect of the invention concerns computer-based methods for determining a representation of a three-dimensional structure of a query protein. These methods typically comprise performing an alignment between the amino acid sequence of a query protein and the amino acid sequences of one or more proteins of known three-dimensional structure to identify a template protein (having a known three-dimensional structure). The structure of the template protein is then used as a template to generate one or more preliminary lattice representations of the three-dimensional structure of the query protein. This lattice representation can be of any aspect of the query protein. For example, it can be a reduced model, e.g., a representation of atoms comprising the polypeptide backbone, side chains, a combination thereof, or any other suitable surrogate for components of the query protein. In certain embodiments, the preliminary representation(s) of the query protein comprises a lattice representation of one or more atoms of the polypeptide's backbone. In certain other preferred embodiments, the preliminary representation(s) of the query protein comprises a lattice representation of the side chains of the amino acids that comprise the protein. In particularly preferred embodiments, one or more of the side chains is represented as a pseudoatom, with pseudoatoms representing side chain centers of mass being especially preferred. If desired, the resulting model can then be refined to generate a plurality of representations of the three-dimensional structure of the of the query protein. If a number of lattice-based three-dimensional reduced models of the query protein's structure are generated, a consensus structure may optionally then be obtained. Additionally, or in the alternative, the representation of the query protein's structure can be further expanded to include representations of one or more non-backbone atoms, up to and including all atoms.

A wide range of embodiments exist for this aspect of the invention. For example, in some embodiments, the alignment between the amino acid sequences of the query and various potential template proteins is performed by any suitable sequence alignment method. Template proteins identified by such methods are preferably those whose amino acid sequences exhibit sufficiently high sequence identity (i.e., a sequence identity of at least about more than 40%, preferably at least about 45-80%) with the amino acid sequence of the query protein. The known three-dimensional structures of those proteins can then be used as templates for constructing three-dimensional model structures for the query protein.

In preferred embodiments, the alignment method used is a sequence-to-structure alignment, i.e., a threading, method. Any suitable threading method may be used. Particularly preferred are threading methods that allow suitable template proteins to be identified when the sequence identity between the amino acid sequences of the query and template protein is less than about 50%, preferably less than about 40% or 35%, and even more preferably less than about 30%, 25%, 20%, 15%, 10%, and even as low as about 5%.

To render the instant invention particularly well suited for maximizing the efficiency of computer resources, in certain preferred embodiments, reduced models of protein structure are generated. For example, in preferred embodiments, the preliminary representation comprises a representation of only one or more backbone atoms (e.g., α-carbon atoms, carboxyl and carbonyl carbon atoms (and their substituent oxygen atom(s)), and amino nitrogen atoms) of each amino acid of the query protein. In other alternative preferred embodiments, the preliminary representation can comprise a representation of the side chains of the amino acids of the query protein, or a combination of representations of the backbone of one or more amino acids and one or more side chains of the query protein. Some of these embodiments include representations of side chains and one or more backbone atoms of one or more of the amino acids of the query protein. In alternative embodiments, representations of backbone atoms of one or more amino acid residues may be replaced by representations of the side chain(s) of the amino acid residues at any appropriate stage in the method. Representations of the side chains, as well as the backbone (or portions of the backbone, for example, the carbon atoms of a given amino acid that form a portion of the backbone), may be of atoms or pseudoatoms (e.g., a center of mass of two or more atoms, for example, a side chain center of mass).

In preferred other embodiments, one or more of the lattice representations is optimized by any suitable method, for example, by Monte Carlo simulations, preferably replica exchange Monte Carlo simulations. In still other embodiments, consensus structures can be calculated when a plurality (i.e., two or more) of lattice representations (including optimized lattice representations) have been generated. Particularly preferred methods for calculating consensus structures include clustering and/or distance geometry methods.

After generating a representation of a three-dimensional structure of a query protein, the representation will typically be stored in any suitable storage device operatively connected to the computer system that generated the representation. The representation may also be visually output on a computer monitor (or device for the visual display of images) operatively connected to the computer system. The resulting image can be used for a variety of purposes, for example, to examine the structure represented, to identify regions of interest in the structure (e.g., structurally distinct domains, regions having particular structures (loops, helices, barrels, turns, etc.), etc.

A related aspect of the invention concerns the three-dimensional structures of query proteins so generated. These structures include those existing in any suitable media, for example, electronic, magnetic, or optical computer storage media, paper or other tangible substrates, as well as those presented transiently (e.g., on a computer monitor). Such structures may be represented in any useful format. Such formats include reduced models, all atom models, space-filling models, ribbon models, “stick and ball” models, etc.

Another aspect of the invention concerns methods of determining a biochemical function for a protein. A representative embodiment of this aspect comprises determining one or more representations of one or more three-dimensional structures of a query (or probe) protein in accordance with the first aspect of the invention. The resulting structural representation(s) can then be probed using one or more functional site descriptors, preferably structure-based functional site descriptors. As functional site descriptor(s) used are correlated with a specific biochemical function (e.g., perform a specific biochemistry, for example, catalyze a particular chemical reaction, bind to a specific type of atom or molecule, etc.), when a match occurs with a particular site in a structural representation of the query protein, the protein is annotated as having the particular function. On the basis of such information, the protein may be screened to identify compounds that modulate (i.e., increase or decrease in a quantitative and/or qualitative manner) the particular function or otherwise specifically interact with the protein, preferably at the functional site corresponding to the functional site descriptor employed. Such screening may be performed in silico, in vitro (for example, in a biochemical assay, e.g., a high throughput biochemical assay), or in vivo. Compounds such as modulators may be provided in any suitable formulation, including formulations suitable for use in assays, pharmaceutical formulations, etc.

Because of the computer-based nature of the methods of the first aspect of the invention, preferred methods for annotating protein function are those that employ large numbers of structural representations (e.g., 10's, 100's, 1,000's, or more), preferably for large numbers of query proteins (e.g., 10's, 100's, 1,000's, 10,000's, 100,000's, or more) that differ in length and/or amino acid sequence by at least one amino acid, probed preferably with a large numbers of functional site descriptors (e.g., 10's, 100's, 1,000's, or more). Thus, using the methods of the invention, it is now possible to perform biochemical function annotation, for example, across some or all of the proteome of a species, provided that the proteome is understood at least at the level of nucleic acid and/or amino acid sequence.

A related aspect is computer program products embodying software for performing the methods of the invention.

Another related aspect of the invention relates to computer systems for implementing the methods of the invention.

Yet another aspect of the invention concerns applications of the invention.

Definitions

The following terms have the following meanings when used herein and in the appended claims. Terms not specifically defined herein have their art recognized meaning, unless another definition is provided elsewhere in the specification, as indicated.

An “amino acid” is a molecule having the structure wherein a central carbon atom (the alpha (α)-carbon atom) is linked to a hydrogen atom, a carboxylic acid group (the carbon atom of which is referred to herein as a “carboxyl carbon atom”), an amino group (the nitrogen atom of which is referred to herein as an “amino nitrogen atom”), and a side chain group, R. When incorporated into a peptide, polypeptide, or protein, an amino acid loses one or more atoms of its amino and carboxylic groups in the dehydration reaction that links one amino acid to another. As a result, when incorporated into a protein, an amino acid is referred to as an “amino acid residue.” In the case of naturally occurring proteins, an amino acid residue's R group differentiates the 20 amino acids from which proteins are synthesized, although one or more amino acid residues in a protein may be derivatized or modified following incorporation into protein in biological systems (e.g., by glycosylation and/or by the formation of cystine through the oxidation of the thiol side chains of two non-adjacent cysteine amino acid residues, resulting in a disulfide covalent bond that frequently plays an important role in stabilizing the folded conformation of a protein, etc.). As those in the art will appreciate, non-naturally occurring amino acids can also be incorporated into proteins, particularly those produced by synthetic methods, including solid state and other automated synthesis methods. Examples of such amino acids include, without limitation, α-amino isobutyric acid, 4-amino butyric acid, L-amino butyric acid, 6-amino hexanoic acid, 2-amino isobutyric acid, 3-amino propionic acid, omithine, norlensine, norvaline, hydroxproline, sarcosine, citralline, cysteic acid, t-butylglyine, t-butylalanine, phenylylycine, cyclohexylalanine, β-alanine, fluoro-amino acids, designer amino acids (e.g., β-methyl amino acids, α-methyl amino acids, Nα-methyl amino acids) and amino acid analogs in general. In addition, when an α-carbon atom has four different groups (as is the case with the 20 amino acids used by biological systems to synthesize proteins, except for glycine, which has two hydrogen atoms bonded to the α-carbon atom), two different enantiomeric forms of each amino acid exist, designated D and L. In mammals, only L-amino acids are incorporated into naturally occurring polypeptides. Of course, the instant invention envisions proteins incorporating one or more D- and L- amino acids, as well as proteins comprised of just D- or L- amino acid residues.

Herein, the following abbreviations may be used for the following amino acids (and residues thereof): alanine (Ala, A); arginine (Arg, R); asparagine (Asn, N); aspartic acid (Asp, D); cyteine (Cys, C); glycine (Gly, G); glutamic acid (Glu, E); glutamine (Gln, Q); histidine (His, H); isoleucine (Ile, I); leucine (Leu, L); lysine (Lys, K); methionine (Met, M); phenylalanine (Phe, F); proline (Pro, P); serine (Ser, S); threonine (Thr, T); tryptophan (Trp, W); tyrosine (Tyr, Y); and valine (Val, V). Non-polar (hydrophobic) amino acids include alanine, leucine, isoleucine, valine, proline, phenylalanine, tryptophan, and methionines. Neutral amino acids include glycine, serine, threonine, cysteine, tyrosine, asparagine, and glutamine. Positively charged (basic amino acids include arginine, lysine and histidine. Negatively charged (acidic) amino acids include aspartic acid and glutamic acid.

A “β-carbon atom” refers to the carbon atom (if present) in the R group of the side chain of an amino acid (or amino acid residue) that is covalently bonded to the α-carbon atom of that amino acid (or residue).

A “pseudoatom” refers to a position in three-dimensional space (represented typically by an x, y, and z coordinate set) that represents the average (or weighted average) position of two or more atoms in a protein or amino acid. Representative examples of a pseudoatom include an amino acid side chain center of mass and the center of mass (or, alternatively, the average position) of an α-carbon atom and the carboxyl atom bonded thereto.

“Protein” refers to any polymer of two or more individual amino acids (whether or not naturally occurring) linked via a peptide bond, and occurs when the carboxyl carbon atom of the carboxylic acid group bonded to the α-carbon of one amino acid (or amino acid residue) becomes covalently bound to the amino nitrogen atom of amino group bonded to the α-carbon of an adjacent amino acid. These peptide bond linkages, and the atoms comprising them (i.e., α-carbon atoms, carboxyl carbon atoms (and their substituent oxygen atoms, with the oxygen atom remaining after formation of the peptide bond being referred to as a “carbonyl oxygen” atom), and amino nitrogen atoms (and their substituent hydrogen atoms)) form the “polypeptide backbone”, or “backbone”, of the protein. In simplest terms, the polypeptide backbone of a protein refers to the amino nitrogen atoms, α-carbon atoms, and carboxyl carbon atoms of each amino acid of the protein, although two or more of these atoms (with or without their substituent atoms) may also be represented as a pseudoatom. Indeed, any representation representing a polypeptide backbone that can be represented in manner that can be manipulated or used computationally will be understood to be included within the meaning of the term “backbone.”

The term “protein” is understood to include the terms “polypeptide” and “peptide” (which, at times, may be used interchangeably herein) within its meaning. Similarly, domains (i.e., discrete portions of a protein that fold independently of the rest of the protein and possesses its own function.) and fragments of proteins and polypeptides are also within the scope of the invention and may be referred to herein as “proteins.”

In a protein, the peptide bonds between adjacent amino acid residues are resonance hybrids of two different electron isomeric structures, wherein a bond between a carbonyl carbon (the carbon atom of the carboxylic acid group of one amino acid after its incorporation into a protein) and a nitrogen atom of the amino group of the α-carbon of the next amino acid places the carbonyl carbon approximately 1.33 Å away from the nitrogen atom of the next amino acid, a distance about midway between the distances that would be expected for a double bond (about 1.25 Å) and a single bond (about 1.45 Å). This partial double bond character prevents free rotation of the carbonyl carbon and amino nitrogen about the bond there between under physiological conditions. As a result, the atoms bonded to the carbonyl carbon and amino nitrogen reside in the same plane, and provide discrete regions of structural rigidity, and hence conformational predictability, in proteins.

Beyond the peptide bond, each amino acid residue contributes two additional single covalent bonds to the polypeptide chain. While the peptide bond limits rotational freedom of the carbonyl carbon and the amino nitrogen of adjacent amino acids, the single bonds of each residue (between the α-carbon and carbonyl carbon (the phi (φ) bond) and between the α-carbon and amino nitrogen (the psi (ψ) bond) of each amino acid), have greater rotational freedom. For example, the rotational angles for φ and ψ bonds for certain common regular secondary structures are listed in the following table:

| | | Approximate | | | |
|---|---|---|---|---|---|
| | | Bond | | | |
| | | Angle | | Residues per | Helix pitch |
| Structure | | φ | ψ | turn | (Å)^{a} |
| Right-handed | α-helix | −57 | −47 | 3.6 | 5.4 |
| (3.6₁₃-helix) | | | | | |

| | | | | |
|---|---|---|---|---|
| 3₁₀-helix | +49 | −26 | 3.0 | 6.0 |
| Parallel β-strand | −119 | +113 | 2.0 | 6.4 |
| Antiparallel β-strand | −139 | +135 | 2.0 | 6.8 |

^{a}helix pitch refers to the distance between repeating turns on a line drawn parallel to the helix axis. Bond angles associated with other secondary structures are known in the art, or can be determined experimentally using standard techniques.

Similarly, the single bond between a α-carbon and its attached R-group provides limited rotational freedom. Collectively, such structural flexibility enables a number of possible conformations to be assumed at a given region within a polypeptide. As discussed in greater detail below, the particular conformation actually assumed depends on thermodynamic considerations, with the lowest energy conformation being preferred.

The particular amino acid sequence of a given protein may be referred to as the polypeptide's “primary structure,” and is typically represented from amino-terminus to carboxy-terminus. In addition to primary structure, proteins also have secondary, tertiary, and, in multi-subunit proteins, quaternary structure. “Secondary structure” refers to local conformation of the polypeptide chain, with reference to the covalently linked atoms of the peptide bonds and α-carbon linkages that string the amino acids of the protein together. Side chain groups are not typically included in such descriptions. Representative examples of secondary structures include α helices, parallel and anti-parallel β structures, and structural motifs such as helix-turn-helix, β-α-β, the leucine zipper, the zinc finger, the β-barrel, and the immunoglobulin fold. Movement of such domains relative to each other often relates to biological function and, in proteins having more than one function, different binding or effector sites can be located in different domains. “Tertiary structure” concerns the total three-dimensional structure of a protein, including the spatial relationships of amino acid side chains and the geometric relationship of different regions of the protein. “Quaternary structure” relates to the structure and non-covalent association of different polypeptide subunits in a multi-subunit protein.

“Proteome” means a set of proteins. A “complete proteome” refers to the entire repertoire of proteins expressed in an organism. As those in the art will appreciate, the proteins expressed in an organism over time will differ. Moreover, the proteins expressed in different organisms of the same species differ due to genetic variation (e.g., alleles, single nucleotide polymorphisms, etc.). A “partial proteome” refers to less than all of the proteins expressed in an organism at a given time. For example, it may refer to the set of proteins expressed at the same time but in different tissues or in the same tissue but at different times in development. As the foregoing makes clear, “proteome” has different meanings, and will depend on the context in which the term is used.

“Sequence identity” or “sequence similarity” means the extent to which two amino acid sequences are invariant.

A “multiple sequence alignment” is an alignment of three or more amino acid sequences with gaps (i.e., spaces introduced into the alignment to compensate for insertions and deletions in one sequence relative to another) inserted in the sequences such that residues with common structural positions and/or ancestral residues are aligned in the same column.

A “reduced model” refers to a three-dimensional structural model of a protein wherein fewer than all heavy atoms (e.g., carbon, oxygen, nitrogen, and sulfur atoms) of the protein are represented. For example, a reduced model might consist of just the α-carbon atoms of the protein, with each amino acid connected to the next amino acid by a virtual bond. Other examples of reduced protein models include those in which only the α-carbon atoms and side chain centers of mass of each amino acid are represented, or where only the polypeptide backbone is represented.

Computational methods usually produce lower quality structures than experimental methods, and the models produced by computational methods are often called “inexact models.” While not necessary in order to practice the instant methods, the precision of these predicted models can be determined using a benchmark set of proteins whose structures are already known. The predicted model for each protein may then be compared to a corresponding experimentally determined structure. The difference between the predicted model and the experimentally determined structure is quantified via a measure called “root mean square deviation” (RMSD). A model having an RMSD of about 2.0 Å or less as compared to a corresponding experimentally determined structure is considered “high quality”. Frequently, predicted models have an RMSD of about 2.0 Å to about 6.0 Å when compared to one or more experimentally determined structures, and are called “inexact models”. As those in the art will appreciate, RMSDs can also be determined for one or more atomic positions when two or experimental structures have been generated for the same protein.

A “functional site” refers to any site in a protein that has confers a function on the protein. Representative examples include active sites (i.e., those sites in catalytic proteins where catalysis occurs), protein-protein interaction sites, sites for chemical modification (e.g., glycosylation and phosphorylation sites), and ligand binding sites. Ligand binding sites include, but are not limited to, metal binding sites, co-factor binding sites, antigen binding sites, substrate channels and tunnels, and substrate binding sites. In an enzyme, a ligand binding site that is a substrate binding site may also be an active site. Functional sites may also be composites of multiple functional sites, wherein the absence of one or more sites comprising the composite results in a loss of function.

Protein structures generated by the instant invention can be of different quality. The highest quality determination methods are experimental structure prediction methods based on x-ray crystallography and NMR spectroscopy. In x-ray crystallography or NMR spectroscopy, “high resolution” structures are those wherein atomic positions are determined at a resolution of about 2 Å or less, and enable the determination of the three-dimensional positioning of each atom (or each non-hydrogen atom) of a protein. “Medium resolution” structures are those wherein atomic positioning is determined at about the 2-4 Å level, while “low resolution” structures are those wherein the atomic positioning is determined in about the 4-8 Å range. Herein, protein structures that have been determined by x-ray crystallography or NMR may be referred to as “experimental structures,” as compared to those determined by computational methods.

As alluded to above, protein structures can also be determined entirely by computational methods, including, but not limited to, homology modeling, threading, and ab initio methods. Often, models produced by such computational methods are “reduced” models, i.e., the predicted structures (or “models”) do not include all non-hydrogen atoms in the protein. Indeed, many reduced models only depict the polypeptide backbone, the side chain centers of mass, etc. of the protein, and such models are preferred in the practice of the invention. Of course, it is understood that once a protein structure based on a reduced model has been generated, all or a portion of it may be further refined to include additional predicted detail, up to including all atom positions.

Computational methods usually produce lower quality structures than experimental methods, and the models produced by computational methods are often called “inexact models.” While not necessary in order to practice the instant methods, the precision of these predicted models can be determined using a benchmark set of proteins whose structures are already known. A computational model may then be compared to a corresponding experimentally determined structure. The difference between the predicted model and the experimentally determined structure may be quantified via any suitable measure. One such measure is called “root mean square deviation” (RMSD). A model having an RMSD of about 2.0 Å or less as compared to a corresponding experimentally determined structure is considered “high quality”. Frequently, predicted models have an RMSD of about 2.0 Å to about 6.0 Å when compared to one or more experimentally determined structures, and are called “inexact models”. As those in the art will appreciate, RMSDs can also be determined for one or more atomic positions when two or experimental structures have been generated for the same protein.

The present invention is particularly useful in deriving structures for proteins that exhibit, at best, only weak sequence identity with proteins whose structures have been determined. This is significant, because at present, for about 30-50% of protein sequences deduced from a given sequenced genome, it is possible to detect only weak sequence identity to proteins of already known structure (Gerstein, M., Proteins 1998, vol. 33:518-34; Hegyi and Gerstein (1999), J. Mol. Biol., vol. 288:147-164; Kelley, et al. (2000), J. Mol. Biol., vol. 299:499-520). The instant methods are particularly useful in enabling the construction of moderate-resolution molecular models for such sequences (as well as for sequences exhibiting greater levels of sequence identity with proteins of known structure), in spite of the often-significant structural differences between the detected template and the actual structure of the query protein, whose three-dimensional structure is unknown.

### BRIEF DESCRIPTION OF THE DRAWINGS

FIG. 1 is a flow diagram showing a generalized overview of a preferred embodiment (sometimes referred to herein as “GENECOMP”) of the comparative modeling methodology of the invention. As indicated, an amino acid sequence of a protein of interest (i.e., the “query sequence”) is carried through a series of steps beginning with a threading-based sequence alignment step, where the query sequence is aligned with one or more template sequences of known structure using a sequence-to-structure method. A multiple sequence alignment step may optionally be included before threading is conducted. If a template sequence is found, the threading step produces a preliminary representation of the three-dimensional structure of the query sequence, as well as predicted contacts and secondary structure that may contain non-aligned regions. In the next step, a lattice representation of the structure of the query sequence is built, after which ab initio folding is performed (preferably in the vicinity of the template structure). Preferred ab initio methods include those that employ a side chain only (“SICHO”) approach to generate a representation of the query sequence's structure. Where the structural representations of the query and template sequences do not align (thereby yielding a region of undefined structure), the ab initio method allows structural models to be created for such regions of undefined structure. Thus, an initial lattice model (termed the “approximate lattice model” in the figure) is produced. Optimization of the lattice model is next carried out, such as by Replica Exchange Monte Carlo (REMC) methodology that takes into account the predicted contact and short-range distance information derived in the course of the threading step, as well various statistical and homology-based potentials. The result of the REMC step is a number optimized lattice models. Thereafter, an average, or consensus, lattice model is calculated by, for example, a clustering or distance geometry procedure. In general, the clustering method has been found to generate better, or higher quality, structural models. If desired, and as illustrated in the figure, the consensus structure (in this embodiment, the structure being represented at this stage only by side chain centers of mass), may then be converted to an all atom model (or model of other varying level of atomic detail, e.g., a backbone only representation) using an appropriate method to generate a representation of the query protein's complete three-dimensional structure.

FIG. 2 shows a plot wherein Root-Mean-Square Deviation (RMSD) of an aligned template region with respect to the native structure of the probe sequence is plotted as a function of the ratio of the number of predicted contacts to the number of residues in the protein. RMSD is plotted on the y axis, while the x axis represents Rc, the ratio of the number of predicted contacts per number of residues. Specifically, the plot shows that good starting models preferably have a value of R_{c} in excess of about 0.5.

FIGS. 3A and 3B are RMSD plots. Plot (A) represents a plot of the RSMD with respect to the native structure of a region of a query sequence aligned by threading to a template versus the RMSD with respect to the native structure of this same region in the lowest energy structure obtained by Monte Carlo simulations. Plot (B) represents a comparison of the RMSD with respect to native of the entire initial structure with the RMSD with respect to native of the entire final structure (i.e., the average structure) extracted from clustering.

FIGS. 4A-F show plots representing lattice simulation trajectories and energy plots (i.e., the trajectories for the lowest energy structure in each case) for three different classes of target sequences (i.e., 1tlk_(A-B), 1tie_(C-D), and 1cid_(E-F)). In the case of 1tlk (FIGS. 4A and B), the RMSD of the initial threading result was good, at 4.61 A. In plot (A), the heavy line represents the RMSD of the whole protein while the light line represents the template region RMSD. In (B) the energy for the simulation carried out in (A) is shown wherein there is little change in overall energy. As shown in plots A and B, where threading results in a good fit, there is little change in the RMSD and energy plots over the course of the trajectory. In the case of 1tie_(FIGS. 4C and D), the RMSD of initial threading result was 7.88 Å. Over the course of the trajectory, plot (C) illustrates that the RMSD of the whole protein (heavy line) and template region (light line) decrease, whereas the energy of the simulation exhibits little change (plot (D)). Plots (E) and (F) relate to the protein 1cid_, which had a less than ideal initial threading-based structure that was 19.8 Å. As shown in plot (E), the RMSD improved little during the course of the trajectory, although the energy for the lowest energy structure in this case dropped during the initial part of the trajectory.

FIGS. 5A-D show representative initial, final, and Modeller structures for four different sequences, 1aba (A), 1rcb (B), 1ten (C), and 3chy (D), respectively. In each structure the native structure is represented by the thin tube, and the predicted structure is represented as the thick tube. In each case, the final predicted structure shows an improvement in RMSD, as compared to the initial model, as well as to the structures produced by Modeller.

Other features and advantages of the invention will be apparent from the following description of the preferred embodiments thereof, and from the claims.

### DETAILED DESCRIPTION

Overview

The present invention provides new and useful comparative modeling methods, a particularly preferred embodiment of which is referred to herein as GENECOMP, which is described in detail below. Among the applications to which the instant methods can be put is to improve the quality of low- to moderate-resolution models of protein structure generated by threading methods, or even other sequence alignment-based methods. In summary, the invention enables determining a representation of at least a portion, and preferably all, of a query protein's three-dimensional structure. Because the methods of the invention can be implemented efficiently via computer, they can be applied rapidly to effect structure determinations for many proteins.

To achieve such results, the computational tools of the invention preferably include the following requirements: an efficient alignment method to detect plausible template proteins of known structure, and to provide an alignment of as good as possible quality. Preferably, a threading algorithm (or a combination of sequence-based and threading approaches) is used. The meaning of “good-quality alignment” in the context of this invention is discussed in detail below. Here, it is important to appreciate that the structural template provided by the alignment of the query sequence to the structure of a known protein does not need to be complete (i.e., a substantial portion (e.g., at least about 25%, 30%, 40%, 50%, 60%, 70%, 80%, 90%, or more) of the target protein(s) may remain undefined). Moreover, the template can differ substantially from the equivalent portion of the (unknown) probe structure, and in many cases, a template/probe RMSD (coordinate Root-Mean-Square Deviation from native after the best superposition) in the range of about 7, 8, 9, 10, 11, 12, 13, 14, or 15 Å is still an acceptable starting conformation.

In addition to an efficient alignment method, a molecular modeling tool that is capable of rearranging the initial alignment-based model in such a way that the final structure is closer (sometimes much closer) to the query protein's “true” structure than it is to the template. This is preferably achieved by employing a modeling tool that allows for the very efficient sampling of protein conformational space (large-scale structural rearrangements) and when the force field of the employed molecular model is capable of selecting native-like structures of the query protein (at least when the search is limited to a portion of conformational space in a neighborhood of the template that is wide enough to comprise the query protein's structure). It is also helpful to be able to a priori estimate the expected accuracy of the obtained molecular models.

To address the issues of fold identification, probe-template alignment, and subsequent refinement, preferred embodiments of the modeling methods of the invention consist of a hierarchy of sequence- and structure-based threading algorithms, Monte Carlo simulations (Kolinski, et al., Proteins 1999, vol. 37:592-610), and distance-geometry-based averaging and/or clustering of the lattice models, optionally coupled with subsequent construction of a detailed atomic model. In spite of the variety of computational tools used, the comparative modeling methods of the invention are quite straightforward, robust, relatively fast, and easy to use in an automated fashion for large-scale protein structure prediction.

It should be noted that the methods of the invention can also be employed in those cases where threading, or a sequence comparison, fails to detect a related global fold (or folds), but indicates possible local structural similarity to protein(s) of known structure. In such cases, a small structural motif (a long helix, helical hairpin, fragment of a β-sheet) can be used as a modeling “template”. Such a template provides a folding scaffold, thereby reducing the conformational space to be searched in order to assemble the remaining portions of the structure of the query protein. Thus there is a continuous transition from the kind of comparative modeling where a structural template having substantial sequence similarity to the query sequence can be identified, through folding in a restricted space around a fragmentary template provided by the threading algorithm to weakly restrained, essentially ab initio folding. As would be expected, the success rate of correct fold assembly and the average accuracy of the obtained molecular models decrease with the decaying quality and length of the template protein alignment. Reasonably good templates enable large, even multi-domain proteins to be modeled, while the “ab initio” dominant approach is limited to small (typically no larger than about 200 residues, preferably no larger than about 170 residues, and more preferably no larger than about 140 residues), single-domain proteins, although for large query proteins, templates that exhibit regions of relatively well-defined alignment as well as even almost no sequence similarity can also be modeled.

This specification describes the invention, as well as its performance using the Fischer database (Fischer, et al. (1996), Pac. Symp. Biocomput., pp. 300-318) as a test set. This test set is commonly used to benchmark threading approaches (see http://www.mbi.ucla.edu/people/fischer/BENCH/benchmark1. hmtl), and is believed to be representative of larger sets of proteins. The similarity level of the related pairs of proteins from this database ranges from closely to very remotely related. Of course, the invention not only enables the detection of related pairs of proteins, but also the ability to obtain good molecular models for a substantial fraction of the test proteins. In this context, perspectives for the large-scale structural (and functional) annotation of proteins encoded in genomic data are also provided. Representative examples of such data includes the genomes (or proteomes) of organisms such as mammals (e.g., humans, cats, dogs, horses, mice, rats, cattle, sheep, goats, and pigs), fish, plants, and pests and pathogens (e.g., viruses, bacteria, etc.), and even from small genomes, such as the genome of M. genitalium (Fraser, et al. (1995), Science, vol. 270:397-403).

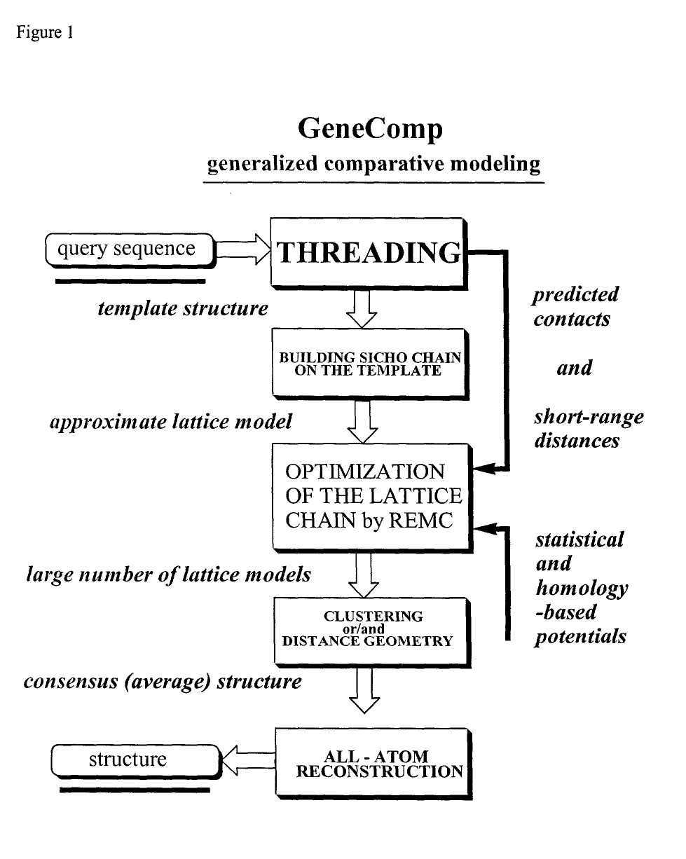

An overview of the model building aspect of the invention is presented in FIG. 1. A brief summary of preferred examples of these methods is provided below, and a number of representative examples of such methods are described in more detail further below.

The Invention

Generally, the present invention applies ab initio folding using a lattice protein model to a preliminary representation of a three-dimensional structure of the query protein generated by a suitable alignment method (or other method of template protein identification), for example, by threading. A particularly preferred threading method useful in the practice of this aspect of the invention is the PROSPECTOR threading method. See PCT patent application US01/30308, filed Sep. 26, 2001, entitled “Methods for Determining Approximations of Three Dimensional Polypeptide Structure”; U.S. provisional patent application No. 60/258,590, filed Dec. 26, 2000, entitled “Novel Threading Methods and Their Use”; U.S. provisional patent application No. 60/235,464, filed Sep. 26, 2000, entitled “Defrosting the Frozen Approximation—PROSPECTOR: A New Approach to Threading”; Skolnick and Kihara, Proteins 2001, vol. ______. As explained in greater detail below, an ab initio folding method is used to refine the preliminary representation in the vicinity of an alignment to a template amino acid sequence. A particularly preferred ab initio folding method is the SICHO (side chain only) method. See U.S. Ser. No. 09/982,488, filed Oct. 17, 2001, and entitled “Protein Modeling Tools”. One or more resulting model structures of the query protein are produced, and if desired, when a plurality of models has been generated, a consensus structure can be derived using any suitable method. Distance geometry methods are among the preferred structure averaging methods, while clustering methods are particularly preferred.

Alignments (Including Threading)

As discussed above, the first step of the methods of the invention concerns identifying one or more template structures that can be used in the generation of one or more preliminary representations of the three-dimensional structure of the query protein. Any approach, whether now known or later developed, that serves this purpose may be employed. In various embodiments of the invention, an alignment method is used. Such methods include multiple sequence alignment methods (e.g., BLAST (basic local alignment search tool; Altschul, et al. (1990), J. Mol. Biol., vol. 215(3):403-410) and PSI-BLAST (position specific iterative BLAST; Altschul, et al. (1997), Nucleic Acids Res., vol. 25(17):3389-3402), although threading methods are preferred.

Among the preferred threading methods useful in practicing the invention is the PROSPECTOR threading method. PROSPECTOR provides predicted contacts and secondary structure not only for the template-aligned regions of the query protein sequence but also, where applicable, for unaligned regions by garnering additional information from other structures. Preferably, this information is incorporated into the refinement algorithm, thereby providing improvement in the unaligned regions as well.

The threading method embodied by PROSPECTOR uses a set of close and distant sequence profiles to generate first pass alignments (Skolnick and Kihara, Proteins 2001, supra). The second pass for each alignment also uses multiple-sequence-averaged pair potentials (interactions between pairs of amino acid residues in a sequence) and secondary structure propensities (terms that reflect the different preferences for local secondary structure (e.g., helices, strands, turns, or loops)), where the partners for the evaluation of the pair interactions are extracted from the alignment generated by the respective first pass sequence profiles. For the top 20 scoring structures (four scoring functions times the best five scoring functions for each structure) if a contact is present in 25% of the structures, then it constitutes a predicted contact. Then, using a previously derived formalism (Skolnick, et al., Proteins 2000, vol. 38:3-16), these predicted contacts are also converted to a threading-based, protein-specific pair potential that is used in a subsequent iteration of threading, designated PROSPECTOR2. Then, additional predicted contacts are collected, and the threading-based, protein-specific pair potential, designated PAIR2, is recalculated using these new contacts. This process is then iterated for a third time, designated PROSPECTOR3. The resulting pair-pair potential is termed PAIR3. The resulting set of contacts from PROSPECTOR1-3 is pooled to form the predicted contacts used in subsequent simulations. On average, PROSPECTOR3 has been found to give the best alignments as well as the best set of predicted local distances. The final template has the best Z-score (where “Z-score” means the energy relative to the mean divided by the standard deviation of the energy) between the distant sequence profile-based threading alignment and the alignment generated from the combined sequence profile, PAIR3, and the secondary structure profile. The entire set of predicted . contacts are used as tertiary restraints. PROSPECTOR3 also provides a set of local chain geometry predictions that are extracted from the average geometry from the top scoring structures and that are subsequently incorporated into the lattice-based folding method.

Multiple sequence alignments and the threading of short test sequence fragments can also be used to derive protein-specific, additional short-range distance restraints (up to several residues along the chain) as well as orientation dependent, protein specific pair potentials.

As those in the art will appreciate, other threading methods, or other sequence-to-structure methods that provide, at a minimum, orientation specific potentials and predicted contacts, may also be adapted for use in conjunction with the instant invention. The goal of this step is to use the query sequence to probe a structural database to derive sequence-specific long-range and short-range potentials. In some cases, the query protein's three-dimensional structural template(s) identified by threading may comprise a plurality of regions representing sufficiently well-defined three-dimensional sub-structures so as to require little or no additional refinement. In other cases, the preliminary representation of the protein's three-dimensional structure identified by in the first step of the method may further comprise one or more of regions representing undefined structures where further refinement would be beneficial.

Lattice Representations

After generating one or more preliminary representations of the three-dimensional structure of the query protein, a refinement procedure is conducted. Preferably, the refinement procedure employs an ab initio folding approach. In preferred embodiments, the refinement procedure involves building at least one lattice representation of at least one of the preliminary representations of the three-dimensional structure of the query protein. In this way, the ab initio folding procedure may provide a refined representation of a three dimensional structure of the query protein. This can be especially useful when in a preliminary representation of a three dimensional structure of the query protein at least one region of the query protein has a less than well-defined structure. The instant refinement process allows one or more sub-structures to be generated that are compatible (i.e., they properly align in structure space, for example, as may be represented as a lattice) with the immediately preceding and/or following regions, as the case may be, of the query protein that have a well-defined sub-structure in the preliminary representation. Of course, this is not to say that such well-defined sub-structures in the preliminary representation can not be further refined by the instant refinement process. After completing these steps, the lattice models is(are) preferably optimized, preferably using a technique such as the Replica Exchange Monte Carlo (REMC) method, which uses the template as a source of weak spatial restraints (Kolinski and Skolnick, Proteins 1998, vol. 32:475-94; Swendsen and Wang (1986), Physical Review Letters, vol. 57:2607-2609).

In preferred embodiments of this part of the invention, proteins are modeled as lattice chains connecting vertices corresponding to the centers of mass of the side chains of the amino acids (such a “side chain center of mass” representation preferably comprises the center of mass of the amino acid's side chain heavy atoms plus the Cα) comprising the query sequence (see, e.g., U.S. Ser. No. 09/982,488, supra). Such models are termed side chain only, or SICHO, models. Preferably, the grid of the underlying simple cubic lattice is equal to 1.45 Å. The distribution of distances between two subsequent chain units mimics the distribution seen in the structural databases of proteins. The various distances reflect the different amino acid sizes, different conformations of the main chain and different side chain rotamers (when applicable). Any given protein structure can be fitted to the corresponding lattice model with an average accuracy of about 0.8 Å RMSD. The model force field contains generic protein-like terms that convert the random coil into an average protein. Included in these generic terms are local stiffness, hydrogen bonding, and side chain packing terms that generate protein-like side chain packing patterns. There are also sequence specific terms that reflect local conformational preferences, local side chain burial, and orientation dependent pair interactions. Knowledge regarding lattice representation of proteins, modeling of protein dynamics, and the basic force field of the model is generally known to those of skill in the art (see, e.g., Kolinski, et al., Proteins 1999, vol. 37:592-610; Skolnick and Kolinski (2001), Adv. Chem. Phys., vol. ______, entitled “A unified approach to the prediction of protein structure and function”; Kolinski and Skolnick, Proteins 1998, vol. 32:475-94).

The alignment of the query sequence to the template structure is usually incomplete and contains insertions and gaps. The building procedure for refining the structural model preferably takes this into consideration. In such preferred embodiments, the aligned residues of the template are first projected onto the lattice, with appropriate (lattice model-based) restrictions for the distances between the two subsequent units and planar angles for three subsequent units. An excluded-volume envelope (the distance of closest approach for two model residues is preferably set at three lattice units, i.e., 4.35 Å) is then built around the alignment. Next, the non-aligned parts of the chains are successively added, taking into account the excluded volume of the already existing chain. In those cases when the number of residues of the query sequence is too small to connect a gap in the template, the closest fragments of the template are relaxed to accommodate such an alignment artifact. Non-aligned ends are attached in a semi-random fashion. For very good alignments, this or like procedures can yield quite accurate lattice models of the query protein. When the alignment becomes worse and covers a small fraction of the query sequence, the starting model may be very far from the target structure.

If desired, models can be optimized by any suitable method. In particularly preferred embodiments that employ lattice models, optimization of lattice folding occurs in a restrained space using a conformational search tool, with the Replica Exchange Monte Carlo method being especially preferred as the conformational search tool (Swendsen, et al. (1986), Physical Review Letters, vol. 57:2607-2609). For this purpose, a number of copies of the initial model are created and placed at various temperatures, according to the REMC scheme. This Monte Carlo simulation consists of two stages. In the first stage, a short annealing run is performed using rather high temperatures in dimensionless units (T=2.5-1.5). Then, in the second stage, the temperature range is set to T=2.0-1.0, and a five-to-ten times longer run compared to the first stage is performed. One simulation trajectory takes 1-2 days on a Pentium III 733 MHz PC. Using twenty copies of the initial model guarantees a very fast and efficient swapping of conformations among the various temperature levels (the temperature increment between replicas has been assumed temperature independent-a linear temperature set), along a lesser or greater number of copies can be used, if desired. Those conformations seen at the lowest temperature of the REMC scheme rapidly find energy minima. The minimum in many cases (as described below) corresponds to near-native conformations. REMC proves to be much more efficient and faster than conventional simulated annealing procedures, and thus is preferred; however, other conformational search tools (whether now known or later developed) may also be employed after appropriate adaptation, as can be performed by one skilled in the art.

The methodologies of the present invention used to improve threading-based protein models is novel in the way the template and other restraints are implemented wherein the template and other restraints use a soft, diverse set of restraints for the lattice folding. In preferred embodiments, three sets of restraints are applied during the folding/optimization procedure. The first set of restraints is associated with the template. For an aligned residue where the equivalence between the target and template residues was established by threading, the following potential is used:

Vₜₑₘₚ₁=V₅+V₁ (1a)

with

V₅ =0 for rₘᵢₙ<2 (in lattice units, or 2.9 Å) (1b)

V₅=0.5·εᵣₑₛₜ·rₘᵢₙ for rₘᵢₙ≧2 (1c)

and

V₁=−εᵣₑₛₜ for r_{i,j}<2 (1d)

where r_{i,j} is the distance between the i-th Cα of the template structure and the jth Cα of the modeled target structure (the first index corresponds to the template residue and the second to the probe, and the aligned pairs have by definition the same indices), and rₘᵢₙ=min{r_{i-2,j}, r_{i-1,j}, r_{i,j}, r_{i+1,j}, r_{i+2,j}}, the smallest distance between the target Cα and the five residue Cα fragments of the template. The last condition allows for 2-residue shifts of the target chains along the template structure, thereby enabling “corrections” of the initial alignment. εᵣₑₛₜ is a constant scaling factor that sets the strength of the restraints. The values of εᵣₑₛₜ as well as the other scaling parameters (see below) may be found in Table I.

TABLE I
Comparison of the accuracy of the models for a “tuning”
set of 12 small proteins produced by GENECOMP with another
generalized comparative modeling technique.^{a}

| | GENECOMP | | | |
|---|---|---|---|---|
| | (PROSPECTOR + | | Other Generalized | |
| | lattice modeling + | | Comparative Modeling | |
| Probe/template proteins | DG averaging) | | Approach | |
| 1aba_/1ego_{—} | 4.75 | (90.8) | 4.86 | (79.3) |
| 1bbhA/2ccy_{—} | 3.07 | (93.9) | 6.82 | (88.5) |
| 1cewI/1molA | 7.79 | (70.4) | 14.38 | (63.9) |
| 1hom_/1lfb_{—} | 1.57 | (97.7) | 3.70 | (58.8) |
| 1stfI/1molA | 7.07 | (69.5) | 5.95 | (84.7) |
| 1tlk_/2rhe_{—} | 3.42 | (95.8) | 4.17 | (83.5) |
| 256bA/1bbh_{—} | 2.44 | (84.9) | 4.36 | (98.0) |
| 2azaA/1paz_{—} | 7.87 | (62.8) | 10.77 | (62.0) |
| 2pcy_/2azaA | 4.03 | (88.9) | 4.41 | (94.5) |
| 2sarA/9rnt_{—} | 5.76 | (91.7) | 7.83 | (76.0) |
| 3cd4_/2rhe_{—} | 7.15 | (92.8) | 6.39 | (85.4) |
| 5fdl_/2fxd_{—} | 11.99 | (55.7) | 12.40 | (65.1) |

^{a}RMSD from native in Å (% of the length of the alignment to the template).

The second set of the restraints originates from the contact prediction procedure. Only a fraction of predicted contacts are exact, i.e., they are native for the target. A much larger fraction of the predicted contacts are almost correct, i.e., they are shifted by ±1 or ±2 residues with respect to the native structure. This was taken into consideration in the design of the restraint potential. Namely,

V_{cont}=−ε_{restc}, for dₘᵢₙ<5 (in lattice units) +ε_{restc}·(dₘᵢₙ−6), for dₘᵢₙ>6 (in lattice units) (2)

where: dₘᵢₙ={r_{i±k,j±k}, k=0,1,2}, and the index (i,j) is the predicted contact in the template structure. The value of the ε_{restc} is scaled relative to the number N_{c} of predicted contacts as follows:

ε_{restc}=εᵣₑₛₜ (for N_{c}<N, where N is the number of residues in the target protein), (3a)

and:

ε_{restc}=εᵣₑₛₜ·N/N_{c} (for N_{c}≧N) (3b)

The positive part of the above potential enters into the total energy when it exceeds a threshold value N_{c}/2, thereby allowing for the significant violation of a small fraction of the restraints.

The third set of restraints contains the target distances predicted from the fragment threading procedure. The corresponding potential could be expressed as follows:

V_{dist}=−ε_{restd}, for |r_{i,j}−R_{i,j|<}1 (in lattice units) +ε_{restd} 4 (|r_{i,j}−R_{i,j}|−1)/(4 +|i−j|) for |r_{i,j}−R_{i,j}|≧1 (in lattice units) (4)

where r denotes the actual distance and R the predicted one. Those terms corresponding to distances that could be larger than the diameter of the target protein, as estimated from the distance |i−j| along the chain, are ignored. Similar to the contact restraints, the strength of the distance restraints is scaled to account for the various numbers, N_{d}, of predicted restraints

ε_{restd=ε}ᵣₑₛₜ (for N_{d}<N, where N is the number of residues in the target protein), (5a)

and

ε_{restd}=εᵣₑₛₜ·N/N_{d} (for N_{d}≧N) (5b)

The positive part of the above potential enters into the total energy when it exceeds a threshold value of N_{d}/5, thereby allowing for the significant violation of a small fraction of the restraints. This reflects the structure of the data for predicted distances, which are similar to the contact-based restraints. Most of them are almost exact.

The total energy of the restraints is the sum of the above-three components for all relevant residues (aligned) or pairs of residues (predicted contacts or distances). There is one adjustable parameter εᵣₑₛₜ in this scheme. The results are not very sensitive to the specific value; however, in the context intrinsic to the model force field, a value of about 0.5 has been determined to be close to optimal when used in the particular preferred embodiment just described.

Consensus and Other Structures

Because the lowest energy structure generated by the refinement simulations may not necessarily be associated with the lowest RMSD from the native structure, it is often desirable to calculate an average, or “consensus” structure, from among the plurality of optimized lattice-based models generated by the preferred refinement step of the methods of the invention. Any suitable averaging method may be employed, but two structure selection protocols are particularly preferred in practicing the methodology of the invention. One such preferred procedure involves calculating the average lattice model by a clustering (see, e.g., Betancourt and Skolnick, J. (2000), J. Comp. Chem., vol. ______, entitled “Finding the needle in a haystack: Educing protein native folds from ambiguous ab initio folding predictions”). The other such preferred procedure is a distance geometry (DG) procedure (see, e.g., Huang, et al. (1999), J. Mol. Biol., vol. 290:267-81). Particularly preferred implementations of clustering and DG methods are described in the four immediately following paragraphs.

Folding simulations near a template can generate clusters of structures with significant differences between them as compared to the differences between the structures within each cluster (see, e.g., Betancourt and Skolnick (2000), J. Comp. Chem., supra). The different clusters, when present, mainly arise from the non-aligned portions in the low energy folds. Therefore, in some preferred embodiments, it is sometimes useful to cluster the structures into groups of different folds prior to obtaining average structures.

When used, the clustering of structures is preferably carried out through a partitioning clustering technique (Betancourt and Skolnick (2000), J. Comp. Chem., supra). As those in the art will appreciate, this method arranges a collection of folds in a multi-dimensional space defined by a metric given by the relative RMSD (RRMSD). The RRMSD is defined as the RMSD divided by a quantity that depends on the radius of gyration of the two structures involved, which when applied to random structures has a mean value approaching one as the chain length increases. The clusters are initially selected by determining the structures with a high probability of being at the center of the cluster, and assigning to them the structures that are significantly close to the center. The centroids for each cluster are determined by optimally aligning the structures in each cluster and then computing their average. The clusters are then refined by an iterative process that consists of centroid calculations followed by the recalculation of the cluster members until a measure of cluster quality is optimized. The resulting clusters are compared to eliminate redundant ones. Finally, the centroid structures are preferably refined by minimizing a harmonic potential constructed from the average distances and standard deviations between every pair of residues for each cluster. This last step is similar to the distance geometry method described below.

For the problem at hand, the structures in each trajectory are clustered to eliminate or reduce unwanted correlations. The resulting centroids from all trajectories are clustered once again to determine the significant folds. Because the structures are significantly similar due to the fact that folding is done in the vicinity of a template, the criterion that determines the size (in terms of RRMSD) of each cluster is determined based on the distribution of the RRMSD between each pair of structures. In particular, two structures with an RRMSD above the average plus two standard deviations of the RRMSD distribution are not allowed in the same cluster. The average energy of the structures of the cluster is assigned to the centroids and used to rank order them.

Again, since the optimal structure prediction in a folding simulation is not always the lowest energy structure, it is preferable to obtain an average or consensus structure among the low-energy folds. An alternative to clustering to improve the force field is distance geometry, which can be implemented in any number of ways. In a representative example, for each of 20 simulations in the second pass described above, 200 (or some lesser or greater number, the exactitude of which is left to the skilled artisan) conformations can be stored in a constant interval of simulation time. The collected structures can then be averaged using a two-step distance geometry, DG, procedure (see Huang, et al. (1999), J. Mol. Biol., vol. 290:267-81). After the first pass, those structures far away from the average are rejected, and the final DG conformation is constructed from the remaining set of structures. Interestingly, DG averaging has been observed to produce a lower RMSD from the native than the average RMSD for the original set of conformations from the lattice simulations. Indeed, the average structures generated by DG have sometimes been observed to be close to the best structures produced by the folding simulations.

Finally, if desired, varying levels of atomic detail, all the way up to an all atom representation, can be added by any suitable method. A fast, representative procedure is as follows that allows for reconstruction of the atomic details from the known positions of the Cαs and the side chains. (If SICHO models are employed, given the side chain center of mass position, this procedure is straightforward as the approximate location of the Cαs can be readily established). The only constraints in this process are the positions of the side chain centers of mass. The initial local Cα trace geometry built as a geometric function of the side chain centers of mass need not be perfect, as the positions of Cαs are optimized in the first step. This is done by a gradient optimization procedure using a very simple force field. There are several harmonic terms in the force field, including the distance between consecutive Cα atoms, the distance between the Cα atom and the side chain center of mass, and a term that regularizes the angular correlation of the Cαs. Thus, an improvement in local geometry occurs. In the next stage, the positions of the backbone atoms are reconstructed according to the local Cα trace conformation. In this step, the vector normal to the plane defined by three consecutive Cαs is calculated. This vector is almost parallel to the peptide bond plane. Thus, the remaining atoms of the peptide bond can be positioned quite accurately. Next, the positions of the side chain atoms are rebuilt. The conformations of the side chains are chosen from a representative rotamer database. For rigid amino acids (e.g., phenylalanine), there is a single conformation in the database. A plurality (e.g., up to 20 or more) of conformations for large, flexible side chains (e.g., lysine) is preferably used. The conformation of the rotamer depends on the distance between the Cα atom and the center of mass of the side chain, and the local chain conformation (e.g., the Cα-Cα-Cα angle). Next, as a final stage of the reconstruction procedure, the side chains are rotated around a virtual Cα center of mass bond to avoid excluded volume conflicts. This procedure yields reasonable structures; however, the packing of side chains after the all-atom reconstruction is not optimized. Such optimization can be readily accomplished using a standard molecular mechanics procedures.

Alternative, but slower, reconstruction methods of comparable accuracy have also been reported (see, e.g., Feig, et al., Proteins 2000, vol. 41:86-97), and while less preferred than the foregoing procedure, may readily be adapted for use in this context.

Automation and Applications

As those in the art will appreciate, this invention is easy to automate, and thus can readily be applied on even whole genome or proteome scales. Automation will occur through the use of computers running computer programs embodying algorithms useful in performing the various procedures described herein.

The methods of the invention will have many uses, as those in the art will appreciate. For example, one application concerns the generation of three-dimensional models of the structure of a query protein. Such models can be stored in any suitable storage media. In some embodiments, the invention further comprises visually outputting the representation of the protein's three-dimensional structure on a computer monitor or other output device, e.g., a printer. The representation can be of different detail, depending on the desired application. For example, for some applications, all atom models are preferred. For other applications, reduced (or less than all atom) models can be used. Examples of reduced models include those wherein only alpha-carbon atoms, beta carbon atoms, all backbone atoms, side-chain center of mass or other pseudoatoms, all non-hydrogen atoms, or combinations of one or more of the foregoing, are represented.

Another important application of the invention concerns methods for determining a biochemical function for a protein. Such methods typically involve determining a representation of at least a portion of a protein's three dimensional structure as described above, followed by probing at least a portion of the representation with one, and preferably a plurality (preferably by computer), of structure-based functional site descriptors to determine if a site in the probed portion of representation matches the structure-based functional site descriptor. Probing structural representations of proteins using one or more structure-based functional site descriptors is described in U.S. patent application Ser. No. 09/322,067, filed May 27, 1999, which methods may be readily adapted to use representations generated by the instant invention.

Still another application for the invention relates to methods of screening for modulator of a structure-correlated biochemical function in a protein, comprising simulating interaction between a test compound model and a representation of the three dimensional structure of a structure in the protein that is correlated with the biochemical function, wherein the structural representation is generated according to the invention. These screening methods are preferably performed via computer. A related aspect concerns modulators identified by such methods, and compositions comprising such modulators.

### EXAMPLES

The following examples are provided to illustrate the practice of preferred embodiments of the instant invention, and in no way limit the scope of the invention.

### Example 1

### Test Proteins

Two sets of proteins were selected for the purpose of tuning and benchmarking the modeling methods of the invention. The first set, containing 12 pairs of target and template proteins, is identical to the set analyzed in previous work on the application of lattice models for the refinement of threading models (Kolinski, et al., Proteins 1999, vol. 37:592-610). For some of these test pairs, the threading method, PROSPECTOR, detects different template structures. However, for the purpose of comparison, the same templates as before were used, regardless of whether their threading scores are lower. The set of 12 proteins was also used to tune the scheme proposed here for the implementation of restraints in the lattice model. The second set of proteins was generated by the threading procedure applied to the Fischer database of sequences and structures (Fischer, et al. (1996), Pac. Symp. Biocomput., pp:300-18).

A. Application to the Set of 12 Proteins and Comparison with Other Work

The test set of 12 proteins was used to “tune” the strength (and their functional form) of various restraints employed in the lattice model. This was done by comparing the average quality of the models resulting from a series of simulations with various scaling factors of particular restraints generated by PROSPECTOR (described above). The results of the modeling for the preferred embodiment of the invention, referred to as GENECOMP, are compared with other generalized comparative modeling results in Table I, above. The GENECOMP models were more accurate (i.e., had lower RMSDs) in 10 of 12 test cases. In five cases, the improvement of the models was of a qualitative nature. Improvement of the models is not only due to refined lattice modeling but also due the DG or Cluster averaging of the final models and due to the (on average) better starting models (alignments to templates) provided by PROSPECTOR.

B. Application to the Fischer Database

The list of 68 target/template protein pairs in the Fischer database is shown in Table II, together with the nomenclature of structure type assigned by SCOP (Fischer, et al., supra).

TABLE II
List of Target/Template protein sets in the Fischer Database

| Target protein | | | Template protein | | |
|---|---|---|---|---|---|
| | | | Structure type by | | |
| PDB code | Name | Length | SCOP | PDB code | Length |
| 1aep_{—} | apolipopholin III | 153 | α; apolipopholin III | 256bA | 106 |
| 1bbhA | cytochrome C | 131 | α; 4 helical up & | 2ccyA | 127 |
| | | | down bundle | | |
| 1bgeB | granulocyte colony- | 159 | α; 4 helical cytokines | 1gmfA | 119 |
| | stimulating factor | | | | |
| 1c2rA | cytochrome C2 | 116 | α; cytochrome | 1ycc | 108 |
| 1cpcL | C-phycocyanin | 172 | α; globin like | 1colA | 197 |
| 1dsbA | disulfide bond | 188 | α; disulfide-bond | 2trxA | 108 |
| | formation protein | | formation facilitator | | |
| | | | (DSBA), insertion | | |
| | | | domain | | |
| 1dxtB | hemoglobin | 147 | α; globin like | 1hbg_{—} | 158 |
| 1hom_{—} | antennapedia | 68 | α; DNA/RNA | 1lfb_{—} | 77 |
| | protein | | binding 3 helical | | |
| | | | bundle | | |
| 1lgaA | lignin peroxidase | 343 | α; heme dependent | 2cyp_{—} | 293 |
| | | | peroxidase | | |
| 1osa_{—} | calmodulin | 148 | α; EF hand like | 4cpv | 108 |
| 1rcb_{—} | interleukin 4 | 129 | α; 4 helical cytokines | 1gmfA | 119 |
| 2hpdA | cytochrome P450 | 457 | α; cytochrome P450 | 2cpp_{—} | 405 |
| 2sas_{—} | sarcoplasmic | 185 | α; EF hand like | 2scpA | 174 |
| | calcium-binding | | | | |
| | protein | | | | |
| 1aaj_{—} | amicyanin | 105 | β; cupredoxins | 1paz_{—} | 120 |
| 1arb_{—} | achromobacter | 263 | β; trypsin like serine | 4ptp_{—} | 223 |
| | protease I | | protease | | |
| 1bbt1 | foot-and-mouth | 186 | β; viral coat & capsid | 2plv1 | 288 |
| | disease virus | | | | |
| 1cauB | canavalin | 184 | β; double stranded β | 1cauA | 181 |
| | | | helix | | |
| 1cid_{—} | CD4 | 177 | β; immunoglobulin | 2rhe_{—} | 114 |
| | | | like β sandwich | | |
| 1fclA | immunoglobulin | 207 | β; immunoglobulin | 2fb4H | 229 |
| | FC fragment | | like β sandwich | | |
| 1ltsD | heat-labile | 103 | β; OB-fold | 1bovA | 69 |
| | enterotoxin | | | | |
| 1mdc_{—} | fatty acid binding | 132 | β; lipocalin | 1ifc_{—} | 131 |
| | protein | | | | |
| 1mup_{—} | major urinary | 157 | β; lipocalin | 1rbp_{—} | 174 |
| | protein | | | | |
| 1pfc_{—} | immunoglobulin | 111 | β; immunoglobulin | 3hlaB | 99 |
| | p/Fc fragment | | like β sandwich | | |
| 1sacA | serum amyloid | 204 | β; conA like | 2ayh_{—} | 214 |
| | component | | lectin/glucanases | | |
| 1ten_{—} | tenascin | 90 | β; immunoglobulin | 3hhrB | 195 |
| | | | like β sandwich | | |
| 1tie_{—} | erythrina trypsin | 166 | β; β trefoil | 4fgf_{—} | 124 |
| | inhibitor | | | | |
| 1tlk_{—} | telokin | 103 | β; immunoglobulin | 2rhe_{—} | 114 |
| | | | like β sandwich | | |
| 2azaA | azurin | 129 | β; cupredoxins | 1paz_{—} | 120 |
| 2afnA | nitrite reductase | 331 | β; cupredoxins | 1aozA | 552 |
| 2fbjL | immunoglobulin | 213 | β; immunoglobulin | 8fabB | 214 |
| | FAB fragment | | like β sandwich | | |
| 2mtaC | methylamine | 147 | β; cupredoxins | 1ycc_{—} | 108 |
| | dehydrogenase | | | | |
| 2omf_{—} | OMPF porin | 340 | β; transmembrane β | 2por_{—} | 301 |
| | | | barrels | | |
| 2pia_{—} | phosphatidylinosito | 321 | β; reductase/isomeras | 1fnr_{—} | 296 |
| | I 3-kinase | | e/elongation factor | | |
| | | | common domain | | |
| 2sga_{—} | proteinase A | 169 | β; trypsin like serin | 4ptp_{—} | 223 |
| | | | proteases | | |
| 2sim_{—} | sialidase | 381 | β; 6 bladed β | 1nsbA | 390 |
| | | | propeller | | |
| 2snv_{—} | sinbdis virus capsid | 151 | β; trypsin like serine | 4ptp_{—} | 223 |
| | protein | | proteases | | |
| 3cd4_{—} | CD4 | 97 | β; immunoglobulin | 2rhe_{—} | 114 |
| | | | like β sandwich | | |
| 3hlaB | class I | 99 | β; immunoglobulin | 2rhe_{—} | 113 |
| | histocompatibility | | like β sandwich | | |
| | antigen A2.1 | | | | |
| 4sbvA | southern bean | 199 | β; viral coat and | 2tbvA | 286 |
| | mosaic virus coat | | capsid | | |
| | protein | | | | |
| 8ilb_{—} | interleukin 1-β | 146 | β; β trefoil | 4fg_{—} | 124 |
| 1aba_{—} | glutaredoxin | 87 | α/β; thioredoxin fold | 1ego_{—} | 85 |
| 1atnA | doxyribonuclease I | 372 | α/β; ribonuclease H- | 1atr_{—} | 383 |
| | | | like motif | | |
| 1chrA | chloromuconate | 370 | α/β; | 2mnr_{—} | 357 |
| | cysloisomerase | | | | |
| 1crl_{—} | lipase | 534 | α/β; α/β hydrolase | 1ede_{—} | 310 |
| 1eaf_{—} | dihydrolipoyl | 243 | α/β; coA dependent | 4cla_{—} | 213 |
| | transacetylase | | acyltransferases | | |
| 1gal_{—} | glucose oxidase | 581 | α/β; FAD/NAD(P) | 3cox_{—} | 500 |
| | | | binding domain | | |
| 1gky_{—} | guanylate kinase | 186 | α/β; p-loop | 3adk_{—} | 194 |
| | | | containing nucleotide | | |
| | | | tryphosphate | | |
| | | | hydrolase | | |
| 1gplA | glutathione | 184 | α/β; thioredoxin fold | 2trxA | 108 |
| | peroxidase | | | | |
| 1hrhA | ribonuclease H | 125 | α/β; ribonuclease H | 1rnh_{—} | 148 |
| | domain of HIV-1 | | like motif | | |
| | reverse | | | | |
| | transcriptase | | | | |
| 1mioC | nitrogenase | 525 | α/β; nitrogenase iron | 3minB | 522 |
| | molybdenium-iron | | molybdenium protein | | |
| | protein | | α and β chains | | |
| 1npx_{—} | NADH peroxidase | 447 | α/β; FAD/NAD(P) | 3grs_{—} | 461 |
| | | | binding domain | | |
| 1tahA | lipase | 318 | α/β; α/β hydrolase | 1tca_{—} | 317 |
| 2ak3A | adenylate inase | 226 | αβ; p-loop | 1gky_{—} | 186 |
| | isoenzyme-3 | | containing nucleotide | | |
| | | | tryphosphate | | |
| | | | hydrolases | | |
| 2cmd_{—} | malate | 312 | α/β; NAD(P) binding | 6ldh_{—} | 329 |
| | dehydrogenase | | Rossmann fold | | |
| 2gbp_{—} | D-galactose/D- | 309 | α/β; periplasmic | 2liv_{—} | 344 |
| | glucose binding | | binding protein like I | | |
| | protein | | | | |
| 2mnr_{—} | mandelate racemase | 357 | α/β; TIM α/β barrel | 4enl_{—} | 436 |
| 3chy_{—} | cheY | 128 | α/β; flavodoxin like | 4fxn_{—} | 138 |
| 3rubL | ribulose 1,5- | 442 | α/β; TIM α/β barrel | 6xia_{—} | 387 |
| | bisphosphate | | | | |
| | carboxilase/ | | | | |
| | oxigenase | | | | |
| 1cewI | cystatin | 108 | α + β; cystatin like | 1molA | 94 |
| 1fxiA | ferredoxin I | 96 | α + β; β-grasp(ubiquit- | 1ubq_{—} | 76 |
| | | | in like) | | |
| 1onc_{—} | P-30 protein | 104 | α + β; Rnase like | 7rsa | 124 |
| 1stfI | inhibitor stefin B | 95 | α/β; cystatin like | 1molA | 94 |
| 2hhmA | inositol | 272 | α/β; sugar | 1fbpA | 316 |
| | monophosphatase | | phosphatases | | |
| 2pna_{—} | phosphatidylinosito | 104 | α/β; SH2 like | 1shaA | 103 |
| | 1 3-kinase | | | | |
| 2sarA | ribonuclease SA | 96 | α + β; micorbial | 9rnt_{—} | 104 |
| | | | ribonucleases | | |
| 5fdl_{—} | ferredoxin | 106 | α + β; ferredoxin like | 2fxb_{—} | 81 |
| 1hip_{—} | oxidized high | 85 | small proteins; HIPIP | 2hipA | 71 |
| | potential iron | | (high potential iron | | |
| | protein | | protein) | | |
| 1isuA | high potential iron- | 62 | small proteins; HIPIP | 2hipA | 71 |
| | sulfur protein | | | | |

This standard database contains a wide variety of structural types: 13 α proteins; 27 β; 18 α/β; α+; and 2 small proteins (which have small secondary structure content). The length of the different proteins in the database varies from 62 to 581 amino acid residues. Note that the template protein that has the best structural superposition in the Fischer Database to the probe structure was always used for all of the probe proteins, even when PROSPECTOR failed to assign the correct template in the first position (PROSPECTOR correctly places 61 of 68 pairs in the top position). The focus is on the correct probe-template pairs to demonstrate the ability of GENECOMP to refine the initial alignments generated by PROSPECTOR.

### Example 2

### Accuracy of Threading-Based Contact Prediction

Table III presents the results of this threading-based approach to contact prediction.

TABLE III
Summary of Contact Prediction Results for the Fischer Database

| Name | N_{c}^{a} | δ = 0^{b} | δ = 1^{b} | δ = 2^{b} | δ = 3^{b} | δ = 4^{b} |
|---|---|---|---|---|---|---|
| 1aaj_{—} | 84 | 0.64 | 0.8 | 0.92 | 0.94 | 0.99 |
| 1aba_{—} | 59 | 0.53 | 0.68 | 0.76 | 0.88 | 0.9 |
| 1aep_{—} | 18 | 0 | 0.06 | 0.22 | 0.5 | 0.67 |
| 1arb_{—} | 11 | 0.36 | 0.82 | 0.82 | 0.91 | 1 |
| 1atnA | 134 | 0.28 | 0.54 | 0.75 | 0.82 | 0.86 |
| 1bbhA | 58 | 0.5 | 0.59 | 0.64 | 0.72 | 0.83 |
| 1bbt1 | 15 | 0.2 | 0.27 | 0.73 | 0.87 | 0.93 |
| 1bgeB | 9 | 0.67 | 0.78 | 1 | 1 | 1 |
| 1c2rA | 88 | 0.65 | 0.83 | 0.91 | 0.95 | 0.95 |
| 1cauB | 94 | 0.56 | 0.74 | 0.83 | 0.9 | 0.93 |
| 1cewI | 34 | 0.44 | 0.71 | 0.76 | 0.85 | 0.88 |
| 1chrA | 279 | 0.52 | 0.77 | 0.89 | 0.95 | 0.98 |
| 1cid_{—} | 36 | 0.42 | 0.56 | 0.72 | 0.83 | 0.89 |
| 1cpcL | 54 | 0.07 | 0.37 | 0.48 | 0.57 | 0.69 |
| 1crl_{—} | 67 | 0.28 | 0.42 | 0.57 | 0.72 | 0.81 |
| 1dsbA | 21 | 0.29 | 0.43 | 0.57 | 0.62 | 0.71 |
| 1dxtB | 132 | 0.67 | 0.81 | 0.83 | 0.95 | 0.98 |
| 1eaf_{—} | 145 | 0.45 | 0.66 | 0.75 | 0.86 | 0.93 |
| 1fclA | 50 | 0.66 | 0.82 | 0.88 | 0.92 | 0.96 |
| 1fxiA | 43 | 0.16 | 0.47 | 0.65 | 0.79 | 0.86 |
| 1gal_{—} | 217 | 0.6 | 0.77 | 0.86 | 0.9 | 0.93 |
| 1gky_{—} | 75 | 0.35 | 0.6 | 0.72 | 0.81 | 0.83 |
| 1gplA | 9 | 0 | 0.11 | 0.33 | 0.56 | 0.78 |
| 1hip_{—} | 52 | 0.62 | 0.75 | 0.87 | 0.9 | 0.94 |
| 1hom_{—} | 47 | 0.49 | 0.66 | 0.85 | 0.91 | 0.96 |
| 1hrhA | 54 | 0.28 | 0.65 | 0.8 | 0.91 | 0.94 |
| 1isuA | 31 | 0.26 | 0.61 | 0.9 | 0.94 | 0.94 |
| 1lgaA | 145 | 0.62 | 0.82 | 0.86 | 0.95 | 0.97 |
| 1ltsD | 31 | 0.16 | 0.45 | 0.77 | 0.87 | 0.9 |
| 1mdc_{—} | 14 | 0.36 | 0.5 | 0.64 | 0.64 | 0.64 |
| 1mioC | 178 | 0.54 | 0.78 | 0.86 | 0.9 | 0.96 |
| 1mup_{—} | 98 | 0.57 | 0.8 | 0.91 | 0.97 | 0.98 |
| 1npx_{—} | 237 | 0.62 | 0.74 | 0.86 | 0.93 | 0.95 |
| 1onc_{—} | 99 | 0.68 | 0.85 | 0.92 | 0.95 | 0.96 |
| 1osa_{—} | 116 | 0.43 | 0.53 | 0.62 | 0.67 | 0.68 |
| 1pfc_{—} | 102 | 0.31 | 0.55 | 0.76 | 0.82 | 0.87 |
| 1rcb_{—} | 41 | 0.46 | 0.66 | 0.78 | 0.85 | 0.98 |
| 1sacA | 67 | 0.16 | 0.31 | 0.45 | 0.54 | 0.66 |
| 1stfI | 22 | 0.45 | 0.45 | 0.64 | 0.77 | 0.82 |
| 1tahA | 12 | 0.42 | 0.92 | 0.92 | 1 | 1 |
| 1ten_{—} | 42 | 0.12 | 0.6 | 0.71 | 0.86 | 0.9 |
| 1tie_{—} | 28 | 0.43 | 0.64 | 0.75 | 0.79 | 0.82 |
| 1tlk_{—} | 65 | 0.74 | 0.83 | 0.94 | 0.97 | 0.98 |
| 2afnA | 31 | 0.06 | 0.35 | 0.45 | 0.68 | 0.71 |
| 2ak3A | 92 | 0.23 | 0.45 | 0.62 | 0.74 | 0.79 |
| 2azaA | 105 | 0.15 | 0.28 | 0.53 | 0.6 | 0.7 |
| 2cmd_{—} | 230 | 0.58 | 0.75 | 0.83 | 0.94 | 0.96 |
| 2fbjL | 47 | 0.55 | 0.7 | 0.81 | 0.89 | 1 |
| 2gbp_{—} | 72 | 0.58 | 0.79 | 0.89 | 0.96 | 0.97 |
| 2hhmA | 47 | 0.4 | 0.66 | 0.77 | 0.83 | 0.96 |
| 2hpdA | 87 | 0.48 | 0.74 | 0.84 | 0.91 | 0.92 |
| 2mnr_{—} | 45 | 0.42 | 0.51 | 0.71 | 0.76 | 0.84 |
| 2mtaC | 1 | 1 | 1 | 1 | 1 | 1 |
| 2omf_{—} | 17 | 0.06 | 0.29 | 0.47 | 0.76 | 0.82 |
| 2pia_{—} | 14 | 0.14 | 0.36 | 0.64 | 0.64 | 0.71 |
| 2pna_{—} | 26 | 0.81 | 0.85 | 0.88 | 0.96 | 0.96 |
| 2sarA | 39 | 0.31 | 0.72 | 0.9 | 0.97 | 0.97 |
| 2sas_{—} | 155 | 0.43 | 0.62 | 0.7 | 0.77 | 0.84 |
| 2sga_{—} | 0 | 0.43 | 0.62 | 0.7 | 0.77 | 0.84 |
| 2sim_{—} | 10 | 0.4 | 0.6 | 0.6 | 0.7 | 0.9 |
| 2snv_{—} | 30 | 0.33 | 0.6 | 0.7 | 0.83 | 0.93 |
| 3cd4_{—} | 99 | 0.42 | 0.58 | 0.74 | 0.78 | 0.83 |
| 3chy_{—} | 33 | 0.36 | 0.73 | 0.82 | 0.88 | 0.97 |
| 3hlaB | 36 | 0.53 | 0.69 | 0.89 | 0.89 | 0.94 |
| 3rubL | 53 | 0.06 | 0.28 | 0.58 | 0.77 | 0.85 |
| 4sbvA | 58 | 0.36 | 0.64 | 0.84 | 0.88 | 0.9 |
| 5fdl_{—} | 32 | 0.09 | 0.34 | 0.53 | 0.75 | 0.75 |
| 8ilb_{—} | 59 | 0.46 | 0.73 | 0.85 | 0.92 | 0.95 |
| average | | 0.41 | 0.61 | 0.75 | 0.83 | 0.88 |

^{a}N_{c} is the total number of predicted contacts.
^{b}Fraction of contacts correctly predicted within δ = ± m residues.

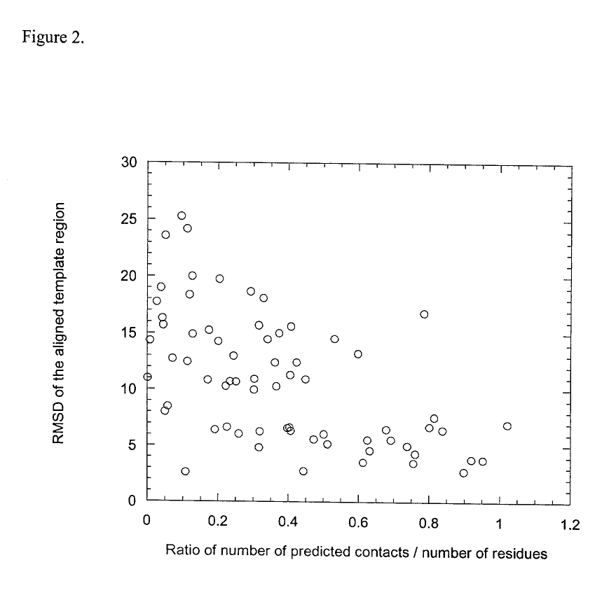

On average, 41% of the contacts were correctly predicted and 75% were correctly predicted within ±2 residues of a correct contact. Since contacts were predicted on an iterative basis, when the fraction of contacts was an appreciable fraction of the entire structure, it was quite likely that the probe-template alignment was significant. This is confirmed in FIG. 2, where the ratio, R_{c}, of the number of contacts to the number of residues exceeds 50%. In 16 of 19 cases, the RMSD of the aligned regions was less than 8 Å and for 9 cases it was less than 5 Å. Thus, good starting models are indicated when R_{c} preferably exceeds about 0.5.

### Example 3

### Optimization of the SICHO Lattice Model by the Replica Exchange Monte Carlo Method

Table IV contains the properties of the starting structures.

TABLE IV
Initial Properties^{a}

| | | | RMSD from | RMSD of | RMSD of |
|---|---|---|---|---|---|
| | | | Threading | Initial | Initial |
| | Number of | Alignment | Result | Structure | Structure |
| Target | Aligned | Coverage | (Aligned | (Aligned | (Whole |
| Protein | Residues | % | Region) | Region) | Protein) |
| 1aaj_{—} | 87 | 82.86 | 6.74 | 8.57 | 13.37 |
| 1aba_{—} | 79 | 90.81 | 6.52 | 6.89 | 7.09 |
| 1aep_{—} | 98 | 64.05 | 18.36 | 18.73 | 18.13 |
| 1arb_{—} | 213 | 80.99 | 16.32 | 16.55 | 17.93 |
| 1atnA | 280 | 75.27 | 12.42 | 12.40 | 14.13 |
| 1bbhA | 123 | 93.89 | 2.74 | 2.99 | 3.34 |
| 1bbt1 | 207 | 97.18 | 12.73 | 10.92 | 10.82 |
| 1bgeB | 110 | 62.86 | 8.50 | 6.02 | 10.29 |
| 1c2rA | 99 | 85.35 | 4.35 | 4.77 | 5.91 |
| 1cauB | 147 | 89.63 | 5.18 | 4.33 | 4.92 |
| 1cewI | 76 | 70.37 | 4.85 | 5.77 | 9.59 |
| 1chrA | 344 | 92.97 | 3.50 | 4.91 | 5.76 |
| 1cid_{—} | 99 | 55.93 | 19.76 | 19.99 | 20.55 |
| 1cpcL | 140 | 81.40 | 15.71 | 15.27 | 15.20 |
| 1crl_{—} | 255 | 47.75 | 20.01 | 20.43 | 23.04 |
| 1dsbA | 94 | 51.65 | 12.46 | 11.77 | 15.39 |
| 1dxtB | 136 | 92.52 | 2.74 | 2.97 | 3.62 |
| 1eaf_{—} | 175 | 78.13 | 13.25 | 11.29 | 11.37 |
| 1fclA | 200 | 96.62 | 12.99 | 13.07 | 13.08 |
| 1fxiA | 59 | 61.46 | 10.94 | 11.34 | 11.84 |
| 1gal_{—} | 430 | 74.01 | 15.03 | 12.45 | 14.59 |
| 1gky_{—} | 159 | 85.48 | 6.68 | 6.70 | 7.86 |
| 1gplA | 104 | 52.79 | 8.03 | 8.31 | 13.63 |
| 1hip_{—} | 68 | 80.00 | 3.55 | 3.71 | 4.92 |
| 1hom_{—} | 43 | 97.73 | 5.56 | 2.92 | 2.94 |
| 1hrhA | 117 | 86.03 | 6.59 | 5.26 | 8.12 |
| 1isuA | 59 | 95.16 | 6.06 | 6.32 | 6.29 |
| 1lgaA | 246 | 77.60 | 12.45 | 10.49 | 14.87 |
| 1ltsD | 59 | 59.00 | 9.99 | 10.29 | 12.65 |
| 1mdc_{—} | 128 | 96.97 | 2.62 | 3.17 | 3.27 |
| 1mioC | 464 | 88.38 | 14.48 | 14.57 | 16.07 |
| 1mup_{—} | 147 | 93.63 | 5.56 | 5.53 | 5.84 |
| 1npx_{—} | 412 | 92.17 | 14.56 | 14.59 | 14.42 |
| 1onc_{—} | 102 | 98.08 | 3.81 | 3.85 | 4.00 |
| 1osa_{—} | 104 | 70.27 | 16.84 | 17.86 | 20.81 |
| 1pfc_{—} | 91 | 89.22 | 3.84 | 5.51 | 6.51 |
| 1rcb_{—} | 92 | 71.32 | 6.28 | 6.13 | 7.17 |
| 1sacA | 156 | 76.47 | 18.13 | 17.89 | 18.93 |
| 1stfI | 75 | 76.53 | 6.66 | 5.55 | 8.52 |
| 1tahA | 181 | 56.92 | 19.00 | 19.36 | 19.93 |
| 1ten_{—} | 84 | 93.33 | 5.60 | 4.70 | 4.79 |
| 1tie_{—} | 103 | 59.88 | 10.85 | 10.97 | 12.49 |
| 1tlk_{—} | 92 | 95.83 | 4.61 | 4.36 | 4.37 |
| 2afnA | 299 | 95.83 | 25.27 | 23.56 | 23.60 |
| 2ak3A | 162 | 78.26 | 15.63 | 19.83 | 19.49 |
| 2azaA | 81 | 62.79 | 7.605 | 8.78 | 11.00 |
| 2cmd_{—} | 299 | 95.83 | 5.016 | 5.24 | 5.43 |
| 2fbjL | 201 | 94.37 | 10.30 | 10.59 | 10.68 |
| 2gbp_{—} | 242 | 80.94 | 10.72 | 10.57 | 12.23 |
| 2hhmA | 195 | 71.69 | 15.26 | 15.81 | 20.24 |
| 2hpdA | 378 | 85.33 | 6.44 | 6.11 | 7.44 |
| 2mnr_{—} | 341 | 95.52 | 14.92 | 15.06 | 15.27 |
| 2mtaC | 96 | 65.31 | 14.35 | 14.53 | 15.68 |
| 2omf_{—} | 279 | 82.06 | 23.61 | 23.69 | 23.47 |
| 2pia_{—} | 255 | 79.44 | 15.72 | 15.88 | 18.03 |
| 2pna_{—} | 27 | 46.55 | 10.69 | 9.19 | 10.50 |
| 2sarA | 88 | 91.67 | 6.36 | 6.51 | 6.94 |
| 2sas_{—} | 160 | 86.49 | 6.45 | 6.88 | 7.56 |
| 2sga_{—} | 179 | 98.90 | 11.02 | 10.92 | 11.78 |
| 2sim_{—} | 252 | 66.14 | 17.74 | 22.18 | 22.29 |
| 2snv_{—} | 127 | 84.11 | 14.28 | 13.64 | 14.70 |
| 3cd4_{—} | 90 | 92.78 | 7.02 | 6.97 | 7.49 |
| 3chy_{—} | 111 | 86.72 | 6.07 | 6.20 | 6.70 |
| 3hlaB | 74 | 83.15 | 10.30 | 10.02 | 9.88 |
| 3rubL | 315 | 66.04 | 24.19 | 23.93 | 24.45 |
| 4sbvA | 194 | 97.49 | 18.68 | 18.82 | 18.75 |
| 5fdl_{—} | 59 | 55.66 | 10.95 | 11.03 | 13.80 |
| 8ilb_{—} | 108 | 73.97 | 11.31 | 12.44 | 13.44 |

^{a}All RMSD values are in Angstroms.

As shown in column three of Table IV, above, although correct templates were used in the initial threading, the alignments did not always cover a large portion of the probe proteins. Indeed, for some cases, this coverage dropped around 50%, explaining why the RMSD of the aligned region from the native structure and that of the whole protein sometimes differed significantly.

The simulation results are summarized in Table V, which shows both the lowest energy structure and the smallest RMSD structure (i.e., the best prediction) in the table, but in what follows, the results of the lowest energy structures are reported unless otherwise indicated, because in general there is no way to estimate what the lowest RMSD structure is in a blind prediction.

TABLE V
Results of Lattice Simulations

| | Lowest Energy Structure | | Smallest RMSD Structure | |
|---|---|---|---|---|
| | RMSD | RMSD | RMSD | RMSD |
| Target | (Whole | (Aligned | (Whole | (Aligned |
| Protein^{a} | Protein) | Region) | Protein) | Region) |
| *1aaj_{—} | 8.42 | 6.17 | 6.15 | 4.92 |
| *1aba_{—} | 5.58 | 5.62 | 3.55 | 3.31 |
| 1aep_{—} | 18.34 | 19.23 | 18.32 | 17.98 |
| 1arb_{—} | 17.30 | 16.62 | 15.78 | 15.78 |
| 1atnA | 13.33 | 12.06 | 12.00 | 11.22 |
| *1bbhA | 3.65 | 3.38 | 2.71 | 2.53 |
| 1bbt1 | 10.81 | 10.92 | 9.57 | 9.33 |
| *1bgeB | 6.27 | 6.02 | 5.04 | 4.93 |
| *1c2rA | 5.37 | 4.77 | 4.31 | 3.85 |
| *1cauB | 5.69 | 4.47 | 4.04 | 3.63 |
| *1cewI | 7.35 | 4.35 | 4.10 | 3.54 |
| *1chrA | 5.11 | 3.79 | 3.77 | 3.36 |
| 1cid_{—} | 18.64 | 18.57 | 14.05 | 13.55 |
| 1cpcL | 13.15 | 13.02 | 12.30 | 12.25 |
| 1crl_{—} | 24.21 | 20.13 | 21.35 | 19.67 |
| 1dsbA | 15.94 | 13.01 | 11.58 | 8.13 |
| *1dxtB | 3.53 | 3.15 | 2.91 | 2.60 |
| 1eaf_{—} | 10.09 | 12.65 | 9.27 | 12.06 |
| 1fclA | 12.89 | 12.97 | 12.43 | 12.50 |
| 1fxiA | 10.28 | 10.80 | 8.53 | 9.20 |
| 1gal_{—} | 17.00 | 14.44 | 14.05 | 11.58 |
| *1gky_{—} | 7.76 | 6.27 | 6.13 | 5.75 |
| 1gplA | 14.75 | 8.98 | 9.08 | 9.63 |
| *1hip_{—} | 4.86 | 4.40 | 3.92 | 3.33 |
| *1hom_{—} | 5.00 | 4.48 | 1.50 | 2.82 |
| *1hrhA | 5.50 | 5.11 | 4.90 | 4.25 |
| *1isuA | 4.23 | 4.31 | 3.20 | 3.19 |
| 1lgaA | 17.13 | 12.51 | 13.10 | 11.81 |
| *1ltsD | 10.25 | 9.95 | 8.11 | 7.79 |
| *1mdc_{—} | 3.12 | 2.95 | 2.55 | 2.51 |
| 1mioC | 15.19 | 14.35 | 14.05 | 13.57 |
| *1mup_{—} | 4.46 | 4.55 | 4.14 | 4.20 |
| 1npx_{—} | 13.75 | 13.97 | 13.61 | 13.76 |
| *1onc_{—} | 3.53 | 3.29 | 3.08 | 3.01 |
| 1osa_{—} | 17.57 | 17.34 | 16.56 | 16.18 |
| *1pfc_{—} | 4.48 | 4.25 | 3.81 | 3.63 |
| *1rcb_{—} | 5.52 | 5.06 | 3.91 | 3.50 |
| 1sacA | 18.21 | 17.43 | 16.89 | 15.51 |
| *1stfI | 7.38 | 4.84 | 4.97 | 4.40 |
| 1tahA | 21.60 | 18.57 | 18.90 | 18.28 |
| *1ten_{—} | 3.96 | 4.01 | 3.16 | 3.17 |
| itie_{—} | 12.88 | 12.82 | 10.74 | 10.73 |
| *1tlk_{—} | 3.19 | 3.33 | 2.35 | 2.35 |
| 2afnA | 23.23 | 24.83 | 22.60 | 24.20 |
| 2ak3A | 15.51 | 15.44 | 14.65 | 14.57 |
| *2azaA | 8.40 | 6.85 | 6.33 | 5.70 |
| *2cmd_{—} | 4.74 | 4.72 | 4.22 | 4.19 |
| 2fbjL | 8.67 | 8.67 | 7.77 | 7.52 |
| 2gbp_{—} | 10.46 | 9.64 | 9.50 | 8.96 |
| 2hhmA | 17.09 | 14.98 | 15.22 | 13.30 |
| *2hpdA | 6.07 | 5.48 | 5.41 | 5.14 |
| 2mnr_{—} | 14.07 | 13.98 | 13.55 | 13.55 |
| 2mtaC | 15.84 | 13.98 | 14.04 | 12.84 |
| 2omf_{—} | 23.51 | 23.06 | 21.82 | 21.99 |
| 2pia_{—} | 17.29 | 15.25 | 15.64 | 14.47 |
| 2pna_{—} | 11.31 | 9.52 | 7.27 | 7.80 |
| *2sarA | 5.93 | 5.70 | 4.88 | 4.77 |
| *2sas_{—} | 5.78 | 5.75 | 5.51 | 5.31 |
| *2sga_{—} | 11.87 | 12.00 | 9.78 | 10.68 |
| 2sim_{—} | 19.79 | 17.06 | 16.52 | 15.50 |
| 2snv_{—} | 13.72 | 13.23 | 12.78 | 12.09 |
| *3cd4_{—} | 7.26 | 6.93 | 5.98 | 5.63 |
| *3chy_{—} | 4.53 | 4.53 | 3.58 | 3.30 |
| 3hlaB | 8.96 | 9.18 | 4.72 | 4.59 |
| 3rubL | 24.19 | 23.72 | 22.26 | 21.73 |
| 4sbvA | 18.12 | 18.19 | 17.73 | 17.75 |
| 5fdl_{—} | 11.64 | 10.52 | 10.70 | 9.34 |
| 8ilb_{—} | 12.58 | 12.01 | 10.77 | 10.59 |

^{a}An asterisk indicates those proteins where the lowest energy structure has an RMSD of less than 10 Å from native. All RMSD values are in Angstroms.

In total, 31 out of 68 targets resulted in structures whose RMSD (whole protein) is less than 10 Å from the native structures. If only the targets with good threading results (those with less than 10 Å RMSD from the native are shown with asterisks in Table V) are counted, 29 of 31 remained less than 10 Å RMSD (exceptions are 1ltsD, which is 10.25 Å and 2sgaA, which is 12.00 Å from native respectively) and 21 resulted in structures whose RMSD from native is less than 6 Å.

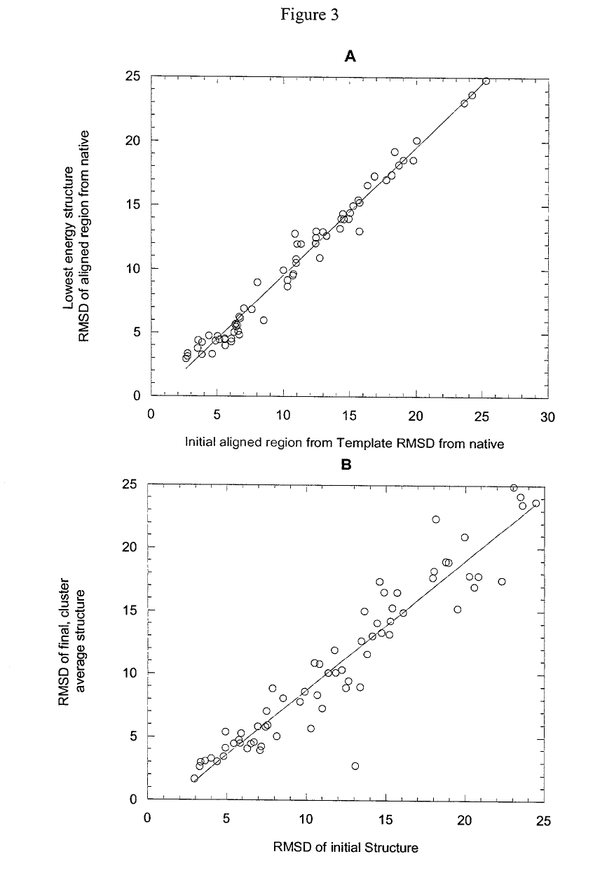

The dependence of the results on the quality of the initial threading alignment is shown in FIGS. 3 A-B. FIG. 3A compares the RMSD of the aligned region obtained from threading with the RMSD of the same region in the lowest energy structure extracted from the simulations. The slope of the best fitting line is 0.96; and some of the lower initial RMSD structures exhibit the most dramatic improvement (see lower left hand corner). FIG. 3B shows the plot of the RMSD of the entire initial structure versus the RMSD of the entire final structure that is extracted from the clustering algorithm. This is well described by a line with a slope of 1.03 and an intercept of −1.56. This curve provides a crude estimate of the likely global RMSD following refinement. On average the structures improve; this is evidenced by the average of the ratio of the final RMSD to the initial RMSD that is 0.86. For those targets with greater than 10 Å RMSD starting structures, 26 out of 37 (70.3%) showed an improvement in terms of the RMSD of the whole protein. On the other hand, for targets with less than 10 Å RMSD starting structures, 27 out of 31 (87.1%) showed some improvement. Especially, 15 of the 16 targets whose RMSD of the starting structure lies between 6 Å and 10 Å showed a positive improvement, and 6 out of 16 improved by greater than 2 Å. Generally, it may be stated that a significant improvement occurs when the threading result is rather poor but not so bad as to be nonsensical. If the initial threading result is very accurate, there is no significant room for improvement. Another important item to note is that in almost all cases, the resulting structure has either improved, or when this is not the case, then the level of deterioration is very small. This observation also holds for the aligned region of the proteins, which are never more than 1 Å worse than their threading results (FIG. 3A). This degradation of structure quality is insignificant and recoverable by the clustering or DG procedure. In this sense, these simulations do no harm to the threading-based structure and can be applied to all situations with impunity.

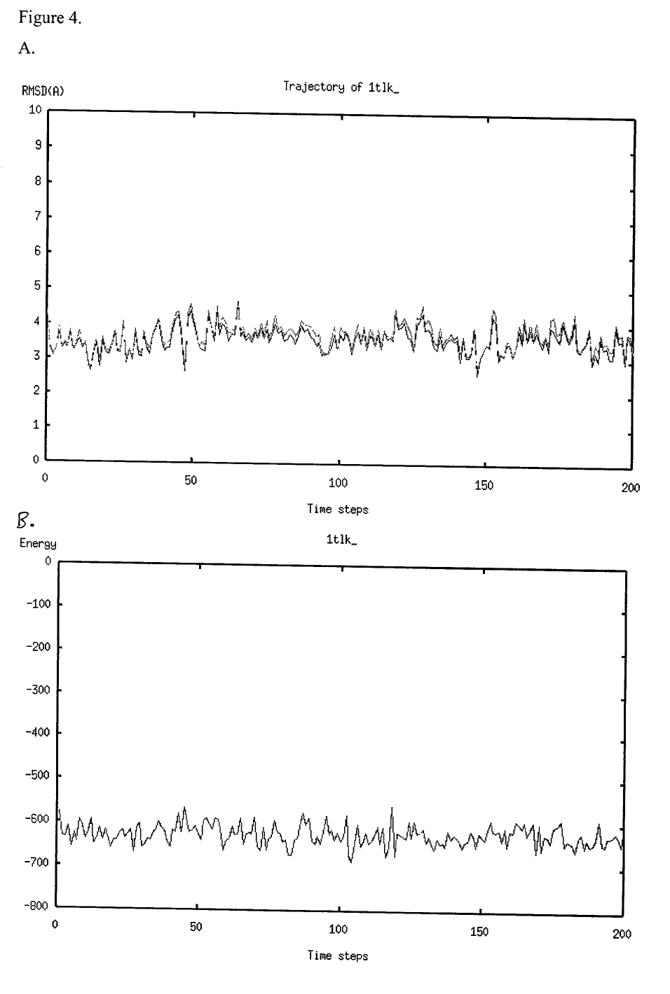

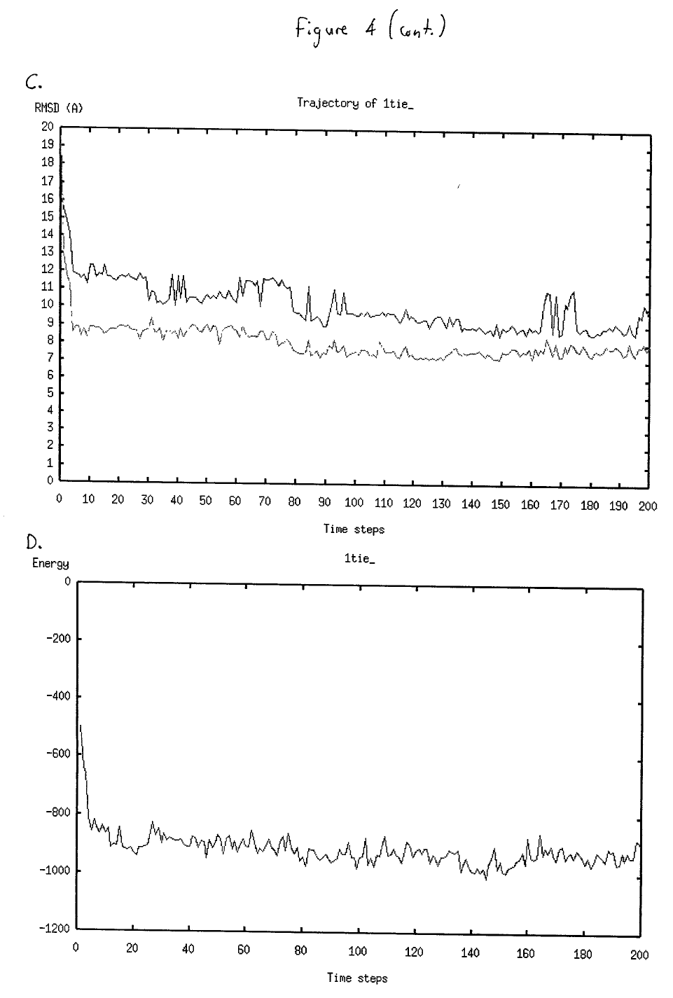

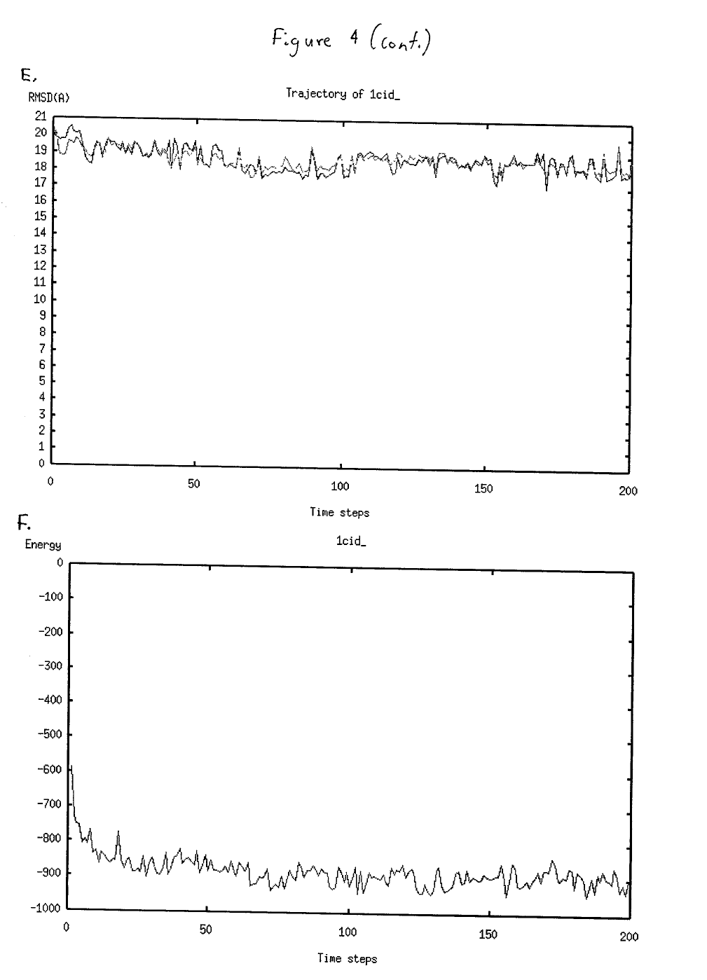

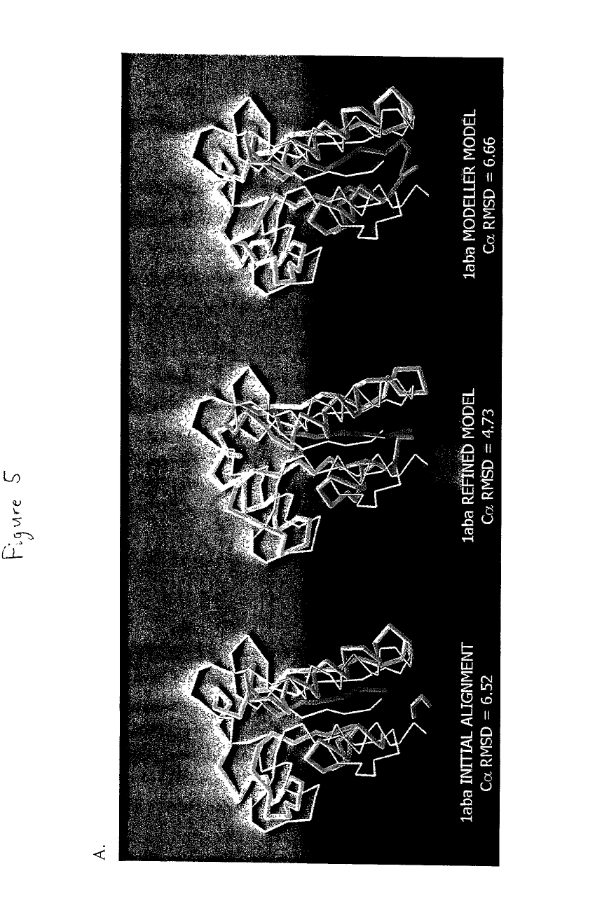

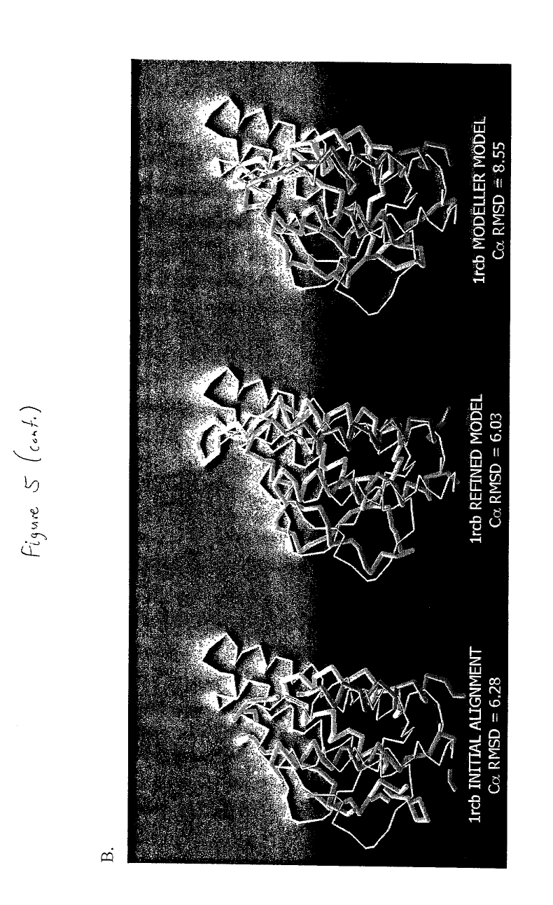

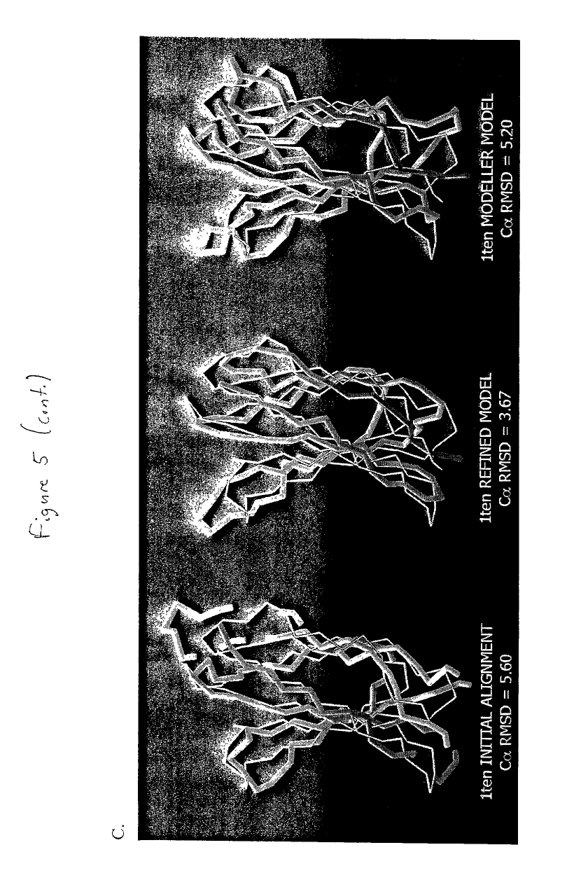

FIGS. 4A-F show typical examples of trajectories of the three classes of targets. When the threading result is good as in FIGS. 4A and B (1tlk_), then both the RMSD and energy don't change significantly during the simulation. Basically, the structure fluctuates near the initial structure. In the case of 1tie_ (FIGS. 4C and D), whose threading result lies in the intermediate range of RMSD, a reduction of both RMSD and energy were observed in the early time steps and the RMSD kept gradually reducing. Generally, this drop of RMSD in the early stage of the simulation corresponds to the relaxation of the initial structure including that of the unaligned region of the threading template. For 1cid_ (FIGS. 4E and F), which has less than ideal initial threading results, the structure did not improve much during the simulation, although the drop of energy in the early stage is still observed. In this particular case, the energy terms describing the generic stiffness, amino acid pair potential, hydrogen bond, and amino acid burial (for detailed explanation of these energy terms, see, for example, Kolinski, et al., Proteins 1999, vol. 37:592-610) are reduced, but this was not enough to drive the structure in the correct direction.

Also investigated was the influence of other possible factors on the simulation results. The first question was whether some structural types of proteins were easier to refine (see Table II, above). It may be worth mentioning that 22 out of 27 β proteins achieved some improvement regardless of the quality of the threading results. Looking at the trajectories of those proteins that exhibited some improvement in spite of a bad initial threading alignment, the RMSD of the final structures were better because of the regularization of β strands and sheets and because they are compact. The number of targets with good results (less than 10 Å RMSD) for each structural type can be readily explained by the fact that most have good initial threading results. There is a weak correlation between the length of the target chain and the final RMSD (the correlation coefficient between protein size and the whole protein RMSD is 0.53). But this is the same range as the correlation between the chain length and RMSD of the initial threading results (0.52). Finally, it was observed that there was no correlation between the number of secondary or tertiary restraints used and the improvement in the RMSD during the simulation.

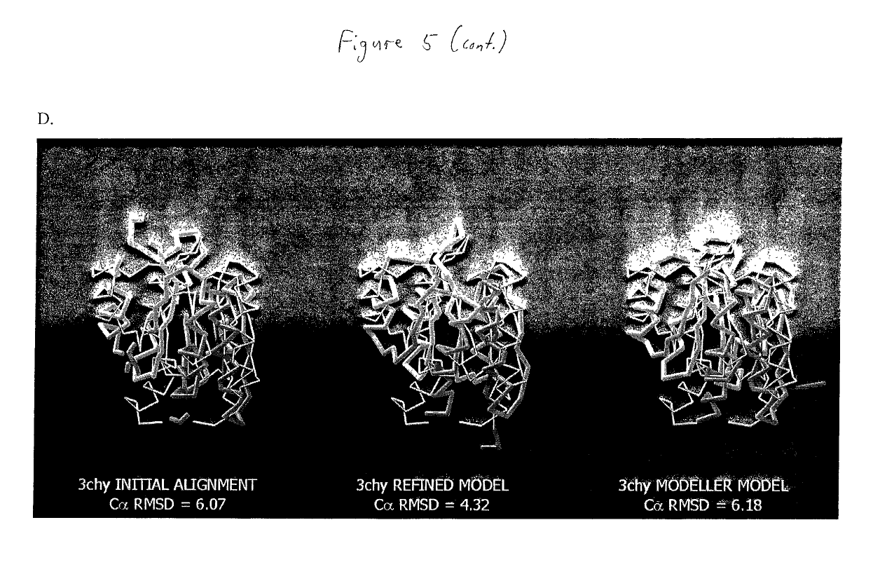

FIGS. 5A-E show a comparison of the initial and final (post distance geometry averaging) structures, as well as the Modeller structures (see below) for proteins 1aba_{—}, 1rcb_{—}, 1ten_, and 3chy_. In all five cases the RMSD of the final structure is lower than that of the initial model, and Modeller is found to perform significantly poorer in this limited set, a point discussed further below. Interestingly, sometimes even for rather good initial structures (e.g. 1onc_) the structures can improve somewhat. Other times, such as in 1rcb_the improvement is minor, but there were cases such as 1aba_{—}, 1ten_and 3chy_where the improvement in the backbone RMSD was on the order of 2 Å.

Turning once again to Table V, none of the lowest energy structures correspond to the smallest RMSD structure. Thus, the potential energy may require improvement. But nonetheless, a practical way to get the best possible structure out of the pool of structures generated by the series of simulations was needed. The following structure refinement procedures were undertaken to achieve this aim.

### Example 4

### Selection of Structures by Distance Geometry.

As shown in Table VI, application of Distance Geometry (DG) with the protocol outlined in the Methods section usually leads to a better structure selection than the structure of lowest conformational energy.

TABLE VI
Comparison of lowest energy, distance geometry generated,
cluster-based, and best possible structures^{a}

| Target | Lowest Energy | DG | Clustering | Best Structure |
|---|---|---|---|---|
| 1aaj_{—} | 8.42 | 9.37 | 9.04 | 6.15 |
| 1aba_{—} | 5.58 | 4.75 | 3.95 | 3.55 |
| 1aep_{—} | 18.34 | 21.45 | 22.38 | 18.32 |
| 1arb_{—} | 17.30 | 17.46 | 17.80 | 15.78 |
| 1atnA | 13.33 | 13.16 | 13.26 | 12.00 |
| 1bbhA | 3.65 | 3.07 | 2.99 | 2.71 |
| 1bbt1 | 10.81 | 10.70 | 10.80 | 9.57 |
| 1bgeB | 6.27 | 5.45 | 5.71 | 5.04 |
| 1c2rA | 5.37 | 5.34 | 5.30 | 4.31 |
| 1cauB | 5.69 | 5.45 | 5.41 | 4.04 |
| 1cewI | 7.35 | 7.79 | 7.85 | 4.10 |
| 1chrA | 5.11 | 4.90 | 4.78 | 3.77 |
| 1cid_{—} | 18.64 | 18.44 | 17.36 | 14.05 |
| 1cpcL | 13.15 | 13.58 | 13.19 | 12.30 |
| 1crl_{—} | 24.21 | 24.09 | 24.93 | 21.35 |
| 1dsbA | 15.94 | 16.47 | 15.90 | 11.58 |
| 1dxtB | 3.53 | 3.01 | 3.08 | 2.91 |
| 1eaf_{—} | 10.09 | 10.32 | 10.10 | 9.27 |
| 1fclA | 12.89 | 13.12 | 13.01 | 12.43 |
| 1fxiA | 10.28 | 10.18 | 10.22 | 8.53 |
| 1gal_{—} | 17.00 | 17.80 | 17.38 | 14.05 |
| 1gky_{—} | 7.76 | 6.36 | 8.94 | 6.13 |
| 1gplA | 14.75 | 13.74 | 15.06 | 9.08 |
| 1hip_{—} | 4.86 | 4.26 | 4.13 | 3.92 |
| 1hom_{—} | 5.00 | 1.57 | 1.70 | 1.50 |
| 1hrhA | 5.50 | 5.07 | 5.07 | 4.25 |
| 1isuA | 4.23 | 5.07 | 4.09 | 3.20 |
| 1lgaA | 17.13 | 15.59 | 16.58 | 13.10 |
| 1ltsD | 10.25 | 10.21 | 9.52 | 8.11 |
| 1mdc_{—} | 3.12 | 2.66 | 2.65 | 2.55 |
| 1mioC | 15.19 | 14.71 | 14.94 | 14.05 |
| 1mup_{—} | 4.46 | 4.38 | 4.51 | 4.14 |
| 1npx_{—} | 13.75 | 14.12 | 14.10 | 13.61 |
| 1onc_{—} | 3.53 | 3.51 | 3.29 | 3.08 |
| 1osa_{—} | 17.57 | 17.90 | 17.85 | 16.56 |
| 1pfc_{—} | 4.48 | 4.28 | 4.46 | 3.81 |
| 1rcb_{—} | 5.52 | 6.09 | 4.30 | 3.91 |
| 1sacA | 18.21 | 18.81 | 19.16 | 16.89 |
| 1stfI | 7.38 | 7.07 | 8.11 | 4.97 |
| 1tahA | 21.60 | 21.51 | 21.47 | 18.90 |
| 1ten_{—} | 3.96 | 3.62 | 3.49 | 3.16 |
| 1tie_{—} | 12.88 | 12.98 | 12.55 | 10.74 |
| 1tlk_{—} | 3.19 | 3.42 | 3.32 | 2.35 |
| 2afnA | 23.23 | 25.05 | 23.55 | 22.60 |
| 2ak3A | 15.51 | 15.46 | 15.29 | 14.65 |
| 2azaA | 8.40 | 7.87 | 7.27 | 6.33 |
| 2cmd_{—} | 4.74 | 4.44 | 4.49 | 4.22 |
| 2fbjL | 8.67 | 8.78 | 8.71 | 7.77 |
| 2gbp_{—} | 10.46 | 10.07 | 10.37 | 9.50 |
| 2hhmA | 17.09 | 17.57 | 17.31 | 15.22 |
| 2hpdA | 6.07 | 5.83 | 5.81 | 5.41 |
| 2mnr_{—} | 14.07 | 14.28 | 14.27 | 13.55 |
| 2mtaC | 15.84 | 16.49 | 16.64 | 14.04 |
| 2omf_{—} | 23.51 | 23.45 | 24.29 | 21.82 |
| 2pia_{—} | 17.29 | 16.77 | 18.41 | 15.64 |
| 2pna_{—} | 11.31 | 8.92 | 10.90 | 7.27 |
| 2sarA | 5.93 | 5.76 | 5.85 | 4.88 |
| 2sas_{—} | 5.78 | 6.11 | 5.95 | 5.51 |
| 2sga_{—} | 11.87 | 10.49 | 11.94 | 9.78 |
| 2sim_{—} | 19.79 | 18.57 | 17.47 | 16.52 |
| 2snv_{—} | 13.72 | 13.84 | 13.38 | 12.78 |
| 3cd4_{—} | 7.26 | 7.15 | 7.05 | 5.98 |
| 3chy_{—} | 4.53 | 4.36 | 4.59 | 3.58 |
| 3hlaB | 8.96 | 8.63 | 8.66 | 4.72 |
| 3rubL | 24.19 | 24.15 | 23.71 | 22.26 |
| 4sbvA | 18.12 | 18.53 | 18.99 | 17.73 |
| 5fdl_{—} | 11.64 | 11.99 | 11.75 | 10.70 |
| 8ilb_{—} | 12.58 | 12.88 | 12.82 | 10.77 |

^{a}All RMSD values are in Angstroms.

In 53 of 68 cases, the structures had a lower RMSD from the native structure after the application of DG. Usually the improvement was small, in the range of 0.3 Å. However, in a number of cases, it was quite significant; in 10 cases the improvement is more than 1.0 Å, and in 4 cases, it is more than 2.0 Å. Only in two cases were the structures after DG significantly worse than the lowest energy structures (more than a 1.0 Å difference).

### Example 5

### Results of Clustering

In general, the comparison of the best centroid to the lowest energy structure only showed a marginal improvement. As shown in Table VI, above, for the 68 structures, the centroid RMSD improved on average by approximately 0.3 Å over the lowest energy structures. Even though in most cases the centroids were similar in quality to the lowest energy structures, only in a few cases were the centroids worse than the lowest energy structures, but in many cases the centroids were significantly better. In 52 of 68 cases, the structure generated by clustering had a lower RMSD from native than the lowest energy structure. Two centroids (1stfI and 1aep) were worse by more than 1 Å as compared to the lowest energy structures, while eight centroids (2fbjL, 1ltsD, 1fc1A, 1aba, 1cid, 1rcb, 3hlaB, 2azaA) were more than 1 Å better than the lowest energy structures. Clustering is clearly the better procedure than distance geometry, as it generates (sometimes only slightly) better structures in 38 cases than distance geometry does, while distance geometry is better in 30 cases. Considering that clustering performs significantly better in ab initio folding, clustering is generally preferred.

### Example 6

### Comparison to Modeller

Recently, several comparative modeling tools have been developed. One of the most widely used is Modeller (Sanchez and Sali, Proteins 1997, suppl:50-8; Sanchez, et al. (2000), Nucleic Acids Res., vol. 28:250-3; Sali and Blundell (1993), J. Mol. Biol., vol. 234:779-815; Sali, et al., Proteins 1995, vol. 23:318-26). Modeller allows for the high-throughput modeling of protein structures on a genomic scale. Since the invention is more complex and more CPU intensive (but high-throughput simulations are certainly possible), the key question is whether GENECOMP performs sufficiently better to justify the increased computational cost. To answer this question, the structures generated by GENECOMP were compared with Modeller in Table VII. Both procedures started from exactly the same templates and the same alignments generated by PROSPECTOR.

TABLE VII
Comparison of Generalized Comparative Modeling with Modeller^{a}

| PROBE | GENECOMP + DG | MODELLER |
|---|---|---|
| 1aaj_{—} | 9.37 | 10.13 |
| 1aba_{—} | 4.75 | 6.66 |
| 1aep_{—} | 21.45 | 21.56 |
| 1arb_{—} | 17.46 | 18.56 |
| 1atnA | 13.16 | 15.61 |
| 1bbhA | 3.07 | 3.02 |
| 1bbt1 | 10.7 | 10.21 |
| 1bgeB | 5.45 | 10.34 |
| 1c2rA | 5.34 | 5.84 |
| 1cauB | 5.45 | 5.93 |
| 1cewI | 7.79 | 8.47 |
| 1chrA | 4.9 | 4.57 |
| 1cid_{—} | 18.44 | 20.19 |
| 1cpcL | 13.58 | 15.62 |
| 1crl_{—} | 24.09 | 25.89 |
| 1dsbA | 16.47 | 16.37 |
| 1dxtB | 3.01 | 3.05 |
| 1eaf_{—} | 10.32 | 10.82 |
| 1fclA | 13.12 | 15.02 |
| 1fxiA | 10.18 | 11.27 |
| 1gal_{—} | 17.8 | 18.86 |
| 1gky_{—} | 6.36 | 11.82 |
| 1gplA | 13.74 | 15.22 |
| 1hip_{—} | 4.26 | 4.06 |
| 1hom_{—} | 1.57 | 1.73 |
| 1hrhA | 5.07 | 6.95 |
| 1isuA | 5.07 | 5.84 |
| 1lgaA | 15.59 | 14.72 |
| 1ltsD | 10.21 | 10.88 |
| 1mdc_{—} | 2.66 | 2.66 |
| 1mioC | 14.71 | 16.78 |
| 1mup_{—} | 4.38 | 4.93 |
| 1npx_{—} | 14.12 | 14.48 |
| 1onc_{—} | 3.51 | 5.14 |
| 1osa_{—} | 17.9 | 16.89 |
| 1pfc_{—} | 4.28 | 4.39 |
| 1rcb_{—} | 6.09 | 8.55 |
| 1sacA | 18.81 | 18.78 |
| 1stfI | 7.07 | 12.76 |
| 1tahA | 21.51 | 23.47 |
| 1ten_{—} | 3.62 | 5.2 |
| 1tie_{—} | 8.6 | 9.3 |
| 1tlk_{—} | 3.42 | 4.31 |
| 2afnA | 25.05 | 25.67 |
| 2ak3A | 15.46 | 19.89 |
| 2azaA | 7.87 | 9.48 |
| 2cmd_{—} | 4.44 | 5.13 |
| 2fbjL | 8.78 | 11.47 |
| 2gbp_{—} | 10.07 | 10.5 |
| 2hhmA | 17.57 | 21.08 |
| 2hpdA | 5.83 | 6.96 |
| 2mnr_{—} | 14.28 | 14.5 |
| 2mtaC | 16.49 | 17.9 |
| 2omf_{—} | 23.45 | 25.34 |
| 2pia_{—} | 16.77 | 16.56 |
| 2pna_{—} | 8.92 | 10.64 |
| 2sarA | 5.76 | 6.59 |
| 2sas_{—} | 6.11 | 6.97 |
| 2sga_{—} | 10.49 | 13.45 |
| 2sim_{—} | 18.57 | 14.43 |
| 2snv_{—} | 13.84 | 12.95 |
| 3cd4_{—} | 7.15 | 7.25 |
| 3chy_{—} | 4.36 | 6.18 |
| 3hlaB | 8.63 | 9.6 |
| 3rubL | 24.15 | 25.6 |
| 4sbvA | 18.53 | 18.93 |
| 5fdl_{—} | 11.99 | 15.03 |
| 8ilb_{—} | 12.88 | 13.76 |

^{a}All RMSD values are in Angstroms.
Note: The same alignments (see Table IV, above) were used as starting templates for GENECOMP and for Modeller.

If all models are considered, then GENECOMP performed better than Modeller in 53 cases, worse in 13, and the same in two cases. Considering only templates whose RMSD is less than 10 Å, then GENECOMP performs better in 29 cases, Modeller performs better in 5 cases, and they perform the same in one case. However, when Modeller does perform better, the two structures differ by a small amount. In many cases of very good (or good) templates, the two methods generate models of similar quality. The situation changes when the level of homology becomes weaker and when, consequently, the threading models are more distant from the probe structure. Here, the models generated by GENECOMP are almost always of noticeably better accuracy. As can easily be seen from the data compiled in Table VII in the range of 4-8 Å RMSD, GENECOMP almost always generated better models than Modeller. The typical difference is 1-2 Å; however, in a few cases it is as much as 4-5 Å. Thus, on average the instant invention leads to qualitatively better molecular models that will have significant consequences for structure-based protein function prediction and other aspects of proteomics.

Conclusion

GENECOMP, a preferred embodiment of the generalized comparative modeling methods of the invention, improves the quality of moderate-resolution threading models. Briefly, the GENECOMP method performs ab initio folding using a lattice protein model, for example, using the SICHO algorithm, in the vicinity of an alignment to a template provided by an alignment method, preferably by a threading algorithm, of which PROSPECTOR is a particularly preferred embodiment. PROSPECTOR also provides predicted contacts and secondary structure not only for the template-aligned regions but also possibly for the unaligned regions by garnering additional information from other structures. This information can be incorporated into the refinement algorithm and can therefore improve the unaligned regions as well. Since the lowest energy structure generated by the simulations does not necessarily have the lowest RMSD from the native structure, two structure selection protocols can be employed: distance geometry and clustering. In the above examples, clustering was found to generate somewhat better quality structures in 38 of 68 cases. The resulting structures can also be converted to atomic-detail models. In general, when applied to the Fischer Database, in a significant number of cases GENECOMP was found to improve upon the initial threading model, sometimes dramatically. The procedure is readily automated and can be implemented on a genomic scale.

The invention has preferred applications. Clearly, the quality of the results depends of the initial alignment quality, with initial template alignments in the range below 10 Å likely to see some improvement in the template alignment. Since the method also has the capacity of improving the quality of unaligned regions, particularly if these are not too long or if there are predicted contacts that are reasonably accurate, then improvement in these regions can be expected. As in ab initio folding for sufficient large inserted regions in general, the quality of the model is intimately tied to the quality of the predicted contacts. However since the lattice model has a resolution of about 3 Å RMSD from native (due to deficiencies in the force field; note that the geometric resolution of the SICHO model is about 0.8 Å), templates below this RMSD are preferably not employed. Also, it would be preferred to distinguish between good and poor templates. That is if one has a very good template, one would want to tightly bind the probe sequence to it, whereas if the template is poor, then it should be loosely bound.

The instant invention, as exemplified by GENECOMP, represents a significant advance in the development of techniques that refine moderate resolution threading models. It will have many applications, for example, to enhance the yield of positives for structure-based functional annotation. Functional site descriptors such as the FFFs developed by Fetrow and Skolnick require that the functional site residues be more or less correctly positioned (the backbone RMSD in the vicinity of the active site should be about 4-5 Å). If not, then a false negative may result. The present invention improves the quality of the alignment and thereby will increase the yield of true positives in biochemical function prediction.

All patents and publications mentioned in this specification are indicative of the levels of skill of those skilled in the art to which the invention pertains. The content of all references, including issued patents, patent applications (whether or not published or expired), scientific papers, and all other documents and electronically available information mentioned or cited herein, in whole or in part, cited in this disclosure are incorporated by reference to the same extent as if each reference had been incorporated by reference in their entirety to the same extent as if each individual publication was specifically and individually indicated to be incorporated by reference, and the right to physically incorporate into this specification any and all materials and information from any such articles, patents, patent applications, and other documents and electronically available information is hereby expressly reserved.

The specific methods and compositions described herein as presently representative of preferred embodiments are exemplary and are not intended as limitations on the scope of the invention. Changes therein and other uses will occur to those skilled in the art which are encompassed within the spirit of the invention are defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations that is not specifically disclosed herein as essential. Thus, for example, in each instance herein, in embodiments of the present invention, any of the terms “comprising,” “consisting essentially of” and “consisting of” may be replaced with either of the other two terms. The terms and expressions that have been employed are used as terms of description and not of limitation, and there is not intention, in the use of such terms and expressions, of excluding any equivalents of the features shown and described or portions thereof, but it is recognized that various modifications are possible within the scope of the invention claimed. Thus, it should be understood that although the present invention has been specifically disclosed by preferred embodiments and optional features, modifications, and variations of the concepts herein disclosed may be resorted to by those skilled in the art, and that such modifications and variations are considered to be within the scope of this invention as defined by the appended claims. Other embodiments are within the following claims.

I claim:

1. A computer-based method for determining a representation of a three-dimensional structure of a query protein, comprising:
(a) performing an alignment using an amino acid sequence of a query protein and a plurality of amino acid sequences of proteins of known three-dimensional structure to identify a template protein;
(b) using the template protein as a template to generate a preliminary representation of the three-dimensional structure of the query protein; and
(c) refining the preliminary representation using a lattice representation to generate a representation of a three-dimensional structure of the protein.

2. A method according to claim 1 wherein the alignment is produced by threading, and wherein the preliminary representation is selected from the group consisting of a representation of the side chains of the amino acids that comprise the query protein and a representation of one or more of each of the atoms that comprise the polypeptide backbone of the query protein.

3. A method according to claim 1 wherein the template protein and the query protein have a sequence identity of less than about 30%.

4. A method according to claim 1 wherein the preliminary representation of the three-dimensional structure of the polypeptide backbone of the query protein comprises a representation of at least one backbone atom from each amino acid residue of the query protein.

5. A method according to claim 1 wherein the preliminary representation of the three-dimensional structure of the polypeptide backbone of the query protein is modified to include a side chain representation of a side chain for at least one amino acid residue of the query protein.

6. A method according to claim 5 wherein the preliminary representation of the three-dimensional structure of the polypeptide backbone of the query protein is modified to include a side chain representation for each of a plurality of side chains of amino acid residues of the query protein.

7. A method according to claim 6 wherein the lattice representation is optimized.

8. A methods according to claim 7 wherein the optimization of the lattice representation is performed by a Monte Carlo simulation.

9. A method according to claim 6 wherein a plurality of lattice representations are generated and then optimized by a Monte Carlo simulation.

10. A method according to claim 9 wherein the representation of the three-dimensional structure of the polypeptide backbone of the query protein is a consensus structure calculated from the optimized lattice representations of the query protein.

11. A method according to claim 1 further comprising step (d), wherein step (d) comprises representing at least one non-backbone atom in the three-dimensional structure of the query protein.

12. A method according to claim 11 wherein the representation of the three-dimensional structure of the query protein is an all atom model.

13. A method according to claim 1 wherein the representation of the three-dimensional structure of the query protein is visually output on a computer monitor.

14. A computer-based method for determining a representation of a three-dimensional structure of a query protein, comprising:
(a) performing a threading alignment between an amino acid sequence of a query protein and a plurality of amino acid sequences of proteins of known three-dimensional structure to identify a template protein;
(b) using the template protein as a template to generate a preliminary representation of the three-dimensional structure of the query protein, wherein the preliminary representation represents a side chain center of mass for each amino acid of the query protein;
(c) refining the preliminary representation to generate a plurality of lattice representations, each of which is then optimized by performing a Monte Carlo simulation; and
(d) determining an average structural representation from the plurality of lattice representations, wherein the average structural representation represents the three-dimensional structure of the query protein.

15. A representation of a three-dimensional structure of a query protein produced in accordance with the method of claim 1.

16. A computer-based method of determining a biochemical function for a protein, comprising:
(a) determining a representation of a three-dimensional structure of the protein using a method according to claim 1; and
(b) probing at least a portion of the representation with a structure-based functional site descriptor to determine if a site in the probed portion of representation matches the structure-based functional site descriptor.

17. A method according to claim 15 wherein a plurality of different structure-based functional site descriptors are used as probes.

18. A computer-based method of screening for a modulator of a structure-correlated biochemical function in a protein, comprising simulating interaction between a test compound model and a representation of the three dimensional structure of a function-conferring structure correlated with said structure-correlated biochemical function, wherein the representation of the three dimensional structure of the protein is produced in accordance with the method of claim 1, and identifying a test compound as a modulator when the test compound model interacts with the function-conferring structure of the protein in a manner indicative of a specific molecular interaction.

19. A modulator identified according to the method of claim 18.
